(19) United States
(12) Patent Application Publication
(10) Pub. No.: US 2014/0378750 A1
(43) Pub. Date: Dec. 25, 2014

(54) UTERINE LAVAGE FOR EMBRYO RETRIEVAL

(71) Applicant: PREVIVO GENETICS, LLC, Piedmont, CA (US)

(72) Inventors: John E. Buster, Providence, RI (US); Moses Cesario, Piedmont, CA (US); Steven Paul Woodard, Cupertino, CA (US)

(21) Appl. No.: 13/924,470

(22) Filed: Jun. 21, 2013

(51) Int. Cl. A61B 17/435

(52) U.S. Cl. CPC A61B 17/435
USPC 600/33

(57) ABSTRACT

A device for recovering one or more blastocysts from a uterus of a human includes an outer guide member for insertion into a cervical canal of the human. The outer guide member includes a distal portion with an activatable seal for isolating the uterus from the external environment. The outer guide member defines a lumen having a longitudinal axis. The device also includes an inner catheter located within the lumen and slidable along the longitudinal axis of the lumen relative to the outer guide member. The inner catheter has a distal tip positionable distally of the seal to extend into the uterus and includes a fluid delivery lumen terminating at a distal fluid delivery port for delivering fluid into the uterus. The inner catheter also includes a flared suction port for aspirating fluid and entrained blastocysts from the uterus

This disclosure is related to U.S. patent application Ser. No. 13/335,170, filed Dec. 22, 2011, titled “RECOVERY AND PROCESSING OF HUMAN EMBRYOS FORMED IN VIVO,” hereby incorporated by reference in its entirety.

### TECHNICAL FIELD

This disclosure relates to uterine lavage.

### BACKGROUND

Uterine lavage for recovery and re-implantation of human embryos from human subjects has been performed for the past three decades. In particular, in-vivo fertilized embryos have been recovered from fertile women and transferred to infertile recipient women, producing donor-to-recipient transplanted human pregnancies. The first reported procedure was performed by a University of Los Angeles team in 1983 and produced a live birth in 1984.

### SUMMARY

In general, in an aspect, at a time when a woman's uterus contains in vivo fertilized preimplantation blastocysts, a seal is provided, between the uterus and the external environment, against flow of fluid from the uterus to the external environment. While the seal is provided, fluid is delivered past the seal and into the uterus. The delivered fluid is withdrawn, with the blastocysts, past the seal and from the uterus to the external environment.

Implementations may include one or more of the following features. The recovered in vivo pre-implantation blastocysts are recovered for genetic diagnosis or genetic therapy or sex determination or any combination of two or more of them. One or more of the blastocysts are returned to the uterus of the woman. The one or more blastocysts are returned to the uterus of the woman without having frozen the blastocysts. The blastocysts resulted from artificial insemination. The blastocysts resulted from causing superovulation in the woman. At least one of the pre-implantation blastocysts is treated. The treating includes gene therapy. The in vivo fertilized preimplantation blastocysts are withdrawn from the uterus with an efficiency of greater than 50%. The in vivo fertilized preimplantation blastocysts are withdrawn from the uterus with an efficiency of greater than 80%. The in vivo fertilized preimplantation blastocysts are withdrawn from the uterus with an efficiency of greater than 90%. The in vivo fertilized preimplantation blastocysts are withdrawn from the uterus with an efficiency of greater than 95%. The embryos are frozen. The delivering or withdrawing or both of the fluid is pulsatile. The fluid is withdrawn while the seal is being provided. The seal enables essentially all of the fluid to be withdrawn. The withdrawing of fluid includes aspirating the fluid from the uterus. Both the delivering and the withdrawing are pulsatile and the pulses of the delivering of the fluid and of the withdrawing of the fluid are coordinated.

In one general aspect, a device for recovering one or more blastocysts from a uterus of a human includes an outer guide member for insertion into a cervical canal of the human. The outer guide member includes a distal portion with an activatable seal for isolating the uterus from the external environment. The outer guide member defines a lumen having a longitudinal axis. The device also includes an inner catheter located within the lumen and slidable along the longitudinal axis of the lumen relative to the outer guide member. The inner catheter has a distal tip positionable distally of the seal to extend into the uterus. The inner catheter includes a fluid delivery lumen terminating at a distal fluid delivery port for delivering fluid into the uterus. The inner catheter also includes a flared suction port for aspirating fluid and entrained blastocysts from the uterus.

Implementations may include one or more of the following features. For example, the inner catheter may include a funnel that defines the flared suction port. The funnel may be configured to expand outward when the inner catheter is extended distally relative to the outer guide member. A cross-sectional area of the flared suction port may be larger than a cross-sectional area of the outer guide member. The flared suction port may be located between the seal and the distal fluid delivery port. The flared suction port may be defined around the fluid delivery lumen. The inner catheter may be steerable. The inner catheter may include a memory-retaining material. Upon extension into the uterus, the inner catheter may be configured to be steered by bending according to a pre-bent shape of the memory-retaining material.

In another general aspect, a system for recovering one or more blastocysts from a uterus of a human includes a device and a controller programmed to cyclically deliver lavage liquid to the uterus via the fluid delivery lumen and apply vacuum to the device from a vacuum source remote from the device. The device includes an outer guide member for insertion into a cervical canal of the human. The outer guide member also includes a distal portion with an activatable seal for isolating the uterus from the external environment, the outer guide member defining a lumen having a longitudinal axis. The device also includes an inner catheter located within the lumen and slidable along the longitudinal axis of the lumen relative to the outer guide member. The inner catheter has a distal tip positionable distally of the seal to extend into the uterus. The inner catheter includes a fluid delivery lumen terminating at a distal fluid delivery port for delivering fluid into the uterus and a flared suction port for aspirating fluid and entrained blastocysts from the uterus.

Implementations may include one or more of the following features. For example, the controller may include a pump for delivering the lavage liquid and a pump for applying the vacuum. The controller may include electro-mechanical means for controlling the delivery of lavage fluid and the application of vacuum. The controller may be programmed to cyclically deliver varying amount of lavage liquid. The system may include a lavage fluid bag for supplying the lavage liquid. The system may include an embryo recovery trap for receiving the aspirated fluid and entrained blastocysts.

In another general aspect, a process for recovering one or more blastocysts from a uterus of a human includes placing a device trans-vaginally into the cervical canal, the device including an outer guide member and an inner catheter located within the outer guide member. The outer guide member includes a seal for isolating the uterus from the external environment. The process also includes advancing the inner catheter relative to the outer guide member positioning a distal region of the inner catheter within the uterus, delivering fluid through the inner catheter to the uterus, and applying a vacuum to the uterus to aspirate fluid and entrained blastocysts from the uterus through a flared suction port.

Implementations may include one or more of the following features. For example, placing the device may include locating the seal in the cervical canal. Locating the seal may include locating the seal between the internal cervical os and the external cervical os such that the seal does not extend into the vagina or the uterus. Advancing the inner catheter may include mechanically agitating an inner surface of the uterus. Advancing the inner catheter may include positioning the distal region of the inner catheter proximate an inner wall of the uterus.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIG. 1 is a side view of a lavage device within a female reproductive tract.

FIG. 2 is a side view of the lavage device.

FIGS. 3a and 3b are cross-sectional views of portions of the lavage device.

FIG. 4 is a close-up perspective view of a distal portion of the lavage device.

FIG. 5 is a side cross-sectional view of a handle portion of the lavage device.

FIG. 6 is a side view of the lavage device in a retracted position.

FIG. 7 is a side view of the lavage device in an extended position.

FIG. 8 is a partially cut side view of a distal portion of the lavage device.

FIG. 9 illustrates a steerable tip of the lavage device in FIG. 7.

FIG. 10 is a perspective view of a distal portion of the lavage device.

FIG. 11 is a side view of the lavage device.

FIG. 12 is a front view of a cervical stop of the lavage device.

FIGS. 13-15 are partially cut side views of the lavage device.

FIG. 16 illustrates the lavage device connected to a control cart.

FIG. 17 is a view of a controller interface portion of the control cart.

FIG. 18 is a flow chart illustrating an example process that uses a lavage system.

FIGS. 19-38 illustrate a lavage process using the lavage device.

FIGS. 39 and 40 are side views of another alternative implementation of the lavage device.

FIGS. 41 and 42 illustrate another alternative lavage process using the lavage device of FIGS. 39 and 40.

FIG. 43 is a side view of an alternative implementation of the lavage device.

FIG. 44 is a side view of another alternative implementation of the lavage device.

Like reference symbols in the various drawings indicate like elements.

### DETAILED DESCRIPTION

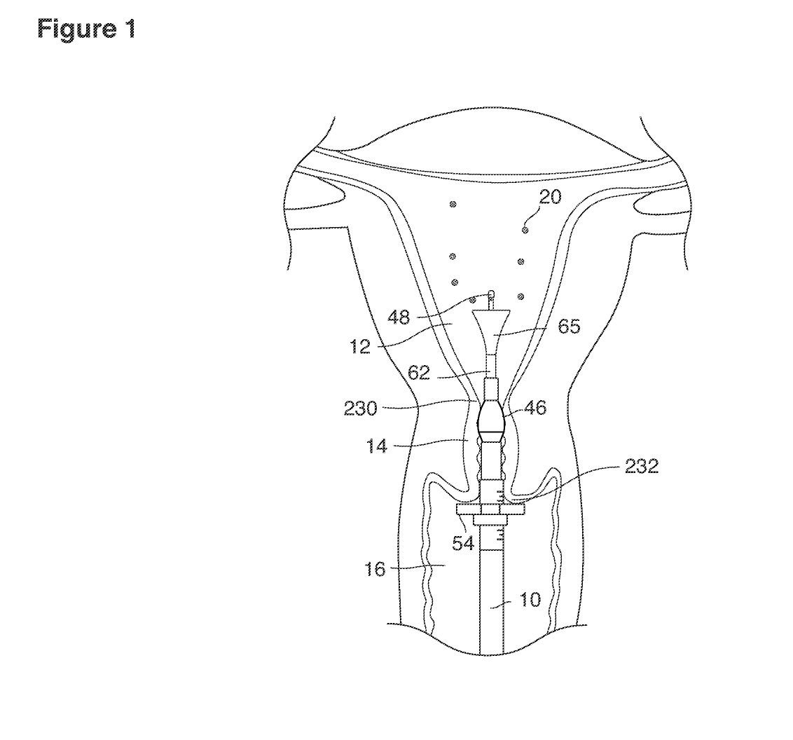

Uterine lavage is performed to withdraw in vivo fertilized preimplantation embryos from a woman. The preimplantation embryos are produced, for example, by superovulation and artificial insemination. Referring to FIG. 1, to perform the uterine lavage, a lavage device 10 is inserted into the uterine cavity 12 via the cervical canal 14 and the vagina 16. The uterine cavity 12 is sealed from the external environment by an activatable seal, for example, an inflatable balloon collar 18 of the lavage device 10, and lavage is performed by introducing fluid into the uterine cavity 12 and withdrawing fluid and entrained preimplantation embryos, i.e., blastocysts 20, from the uterine cavity 12.

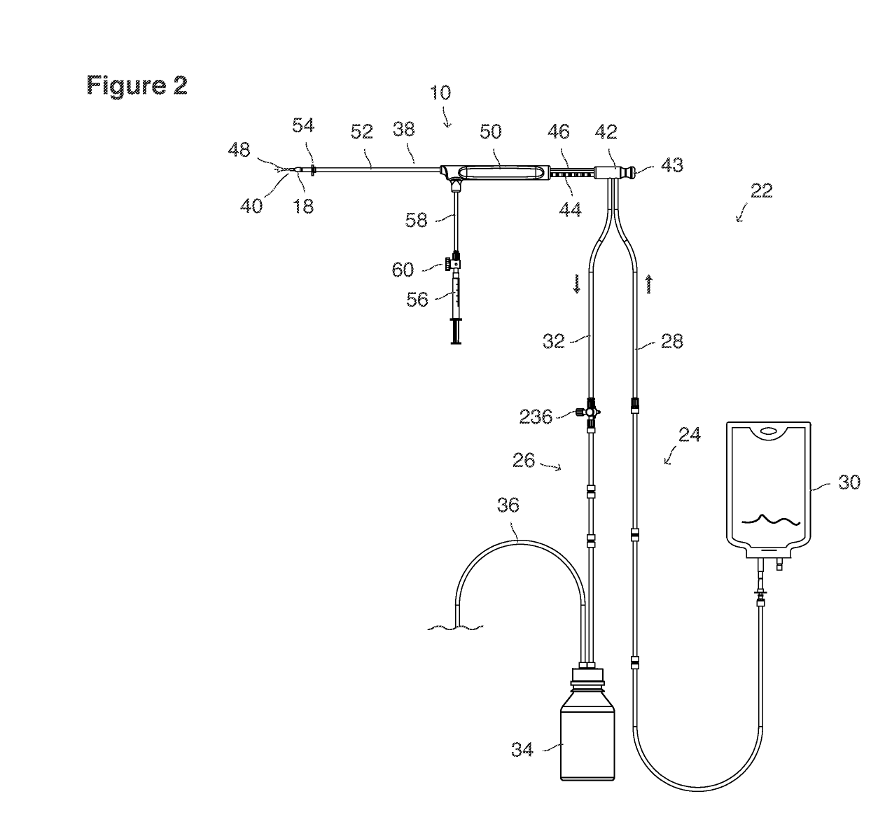

Referring to FIG. 2, a uterine lavage system 22 includes the lavage device 10, an inflow section 24, and an outflow section 26. The inflow section 24 includes a fluid supply line 28 attached to a fluid bag 30, and the outflow section 26 includes a suction recovery line 32 attached to an embryo recovery trap 34, which is attached to a suction line 36. The lavage device 10 includes an outer, formable guide member 38 and an inner catheter 40 slidably received within the outer guide member 38.

The inner catheter 40 includes a manifold 42 to which the fluid supply line 28 and the suction recovery line 32 are attached. The manifold 42 has a control knob 43 for manipulating the inner catheter 40, and extending distally from the manifold 42, the inner catheter 40 includes a stabilizing bar 44, a supply/suction line 46, and an atraumatic tip 48. The outer guide member 38 includes a handle 50, a guide arm 52, a cervical stop 54, and a seal, for example, the balloon collar 18. The balloon collar 18 is inflated using air or liquid delivered by a supply syringe 56 through a supply line 58 attached to the handle 50. Fluid flow through supply line 58 is controlled by a stopcock 60.

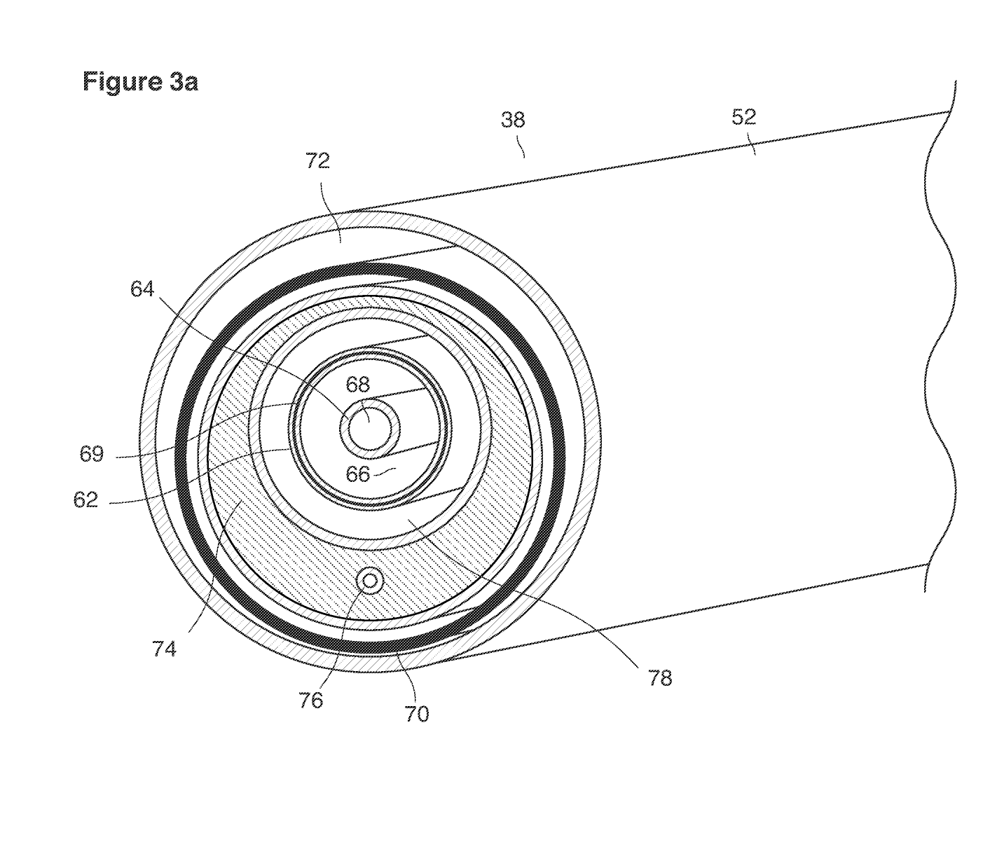

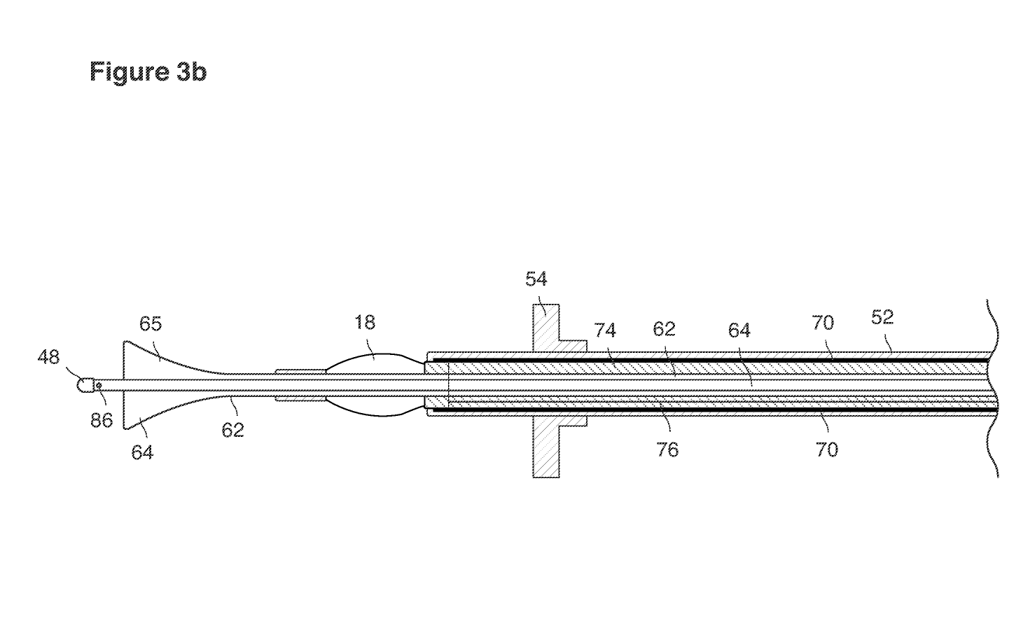

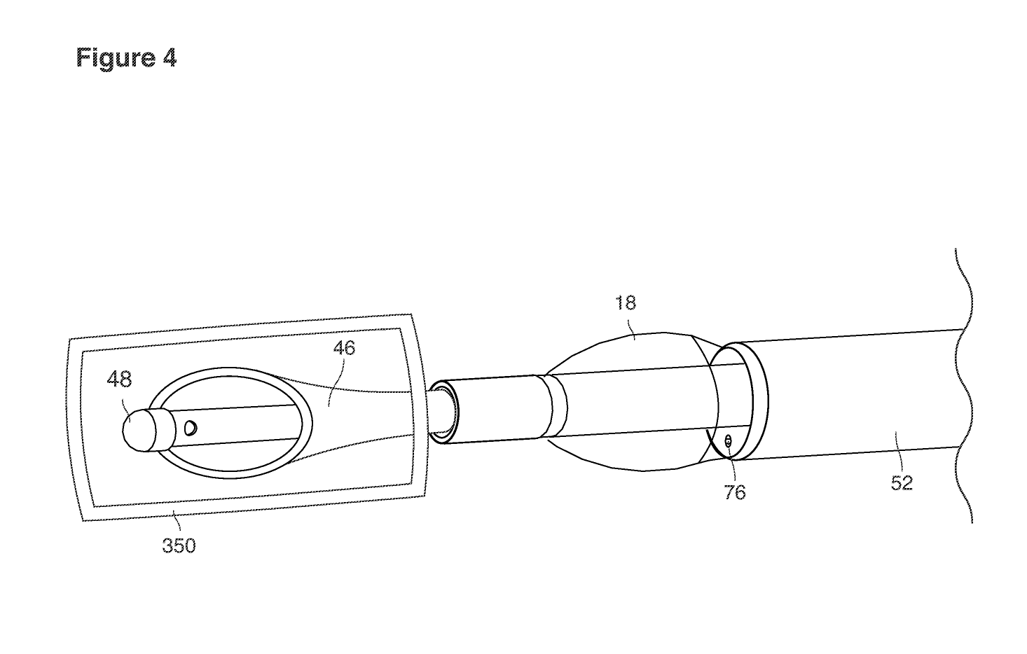

Referring to FIGS. 3A and 3B, the supply/suction line 46 of the inner catheter 40 is a coaxial tube including an outer tubular member 62 and an inner tubular member 64. The inner tubular member 64 terminates in an expandable funnel 65. Defined between the tubular members 62, 64 is an outflow lumen 66 for aspiration of fluid and entrained blastocysts from the uterine cavity, and the inner tubular member 64 defines an inflow lumen 68 for delivery of lavage fluid to the uterine cavity. The outer tubular member 62 includes a formable member 69 that allows the tubular member 62 to be manipulated within the uterine cavity 12, as discussed further below. The outer guide member 38 includes a formable tube 70 located within a lumen 72 of the guide arm 52. The formable tube 70 surrounds a support member 74, which defines a lumen 76 connected to the balloon inflation supply line 58. FIG. 4 shows the termination of the supply lumen 76 at the balloon collar 18. Support member 74 defines a lumen 78 (FIG. 3a) that receives the supply/suction line 46 of the inner catheter 40.

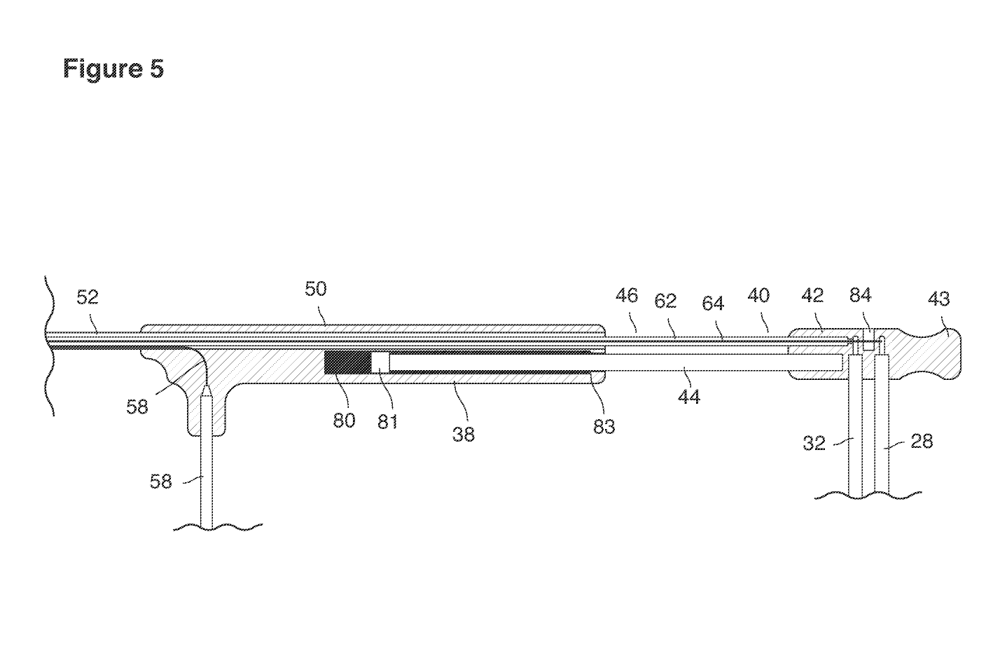

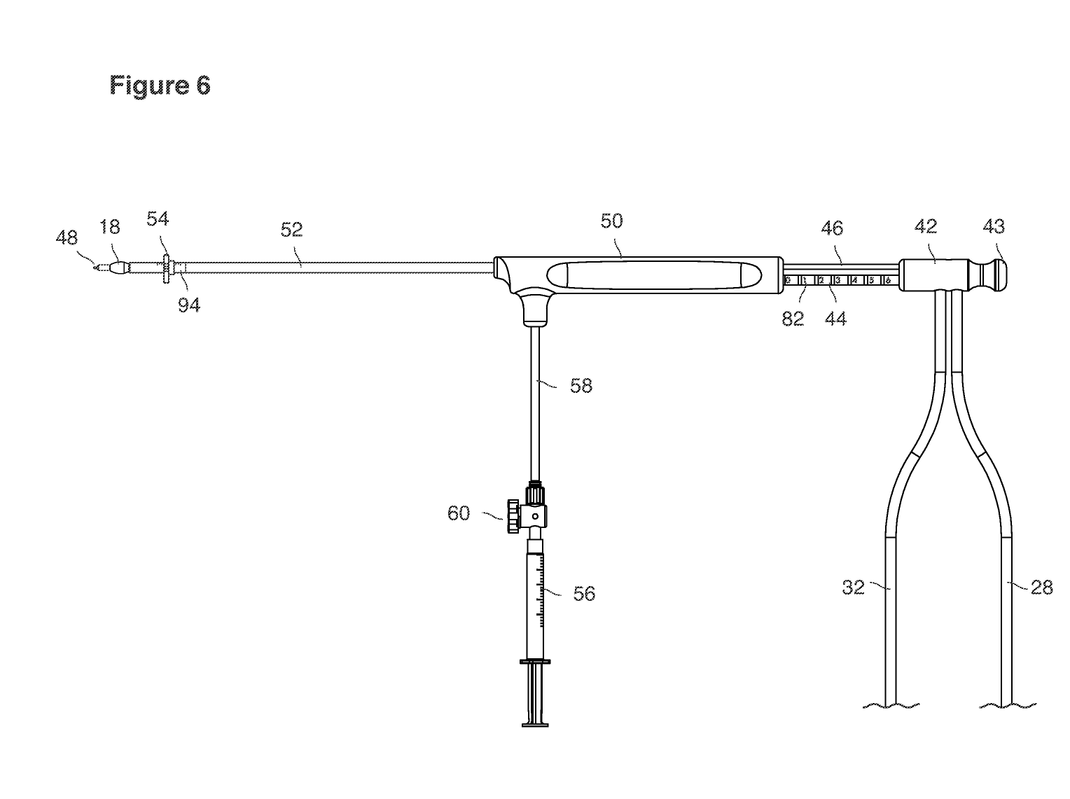

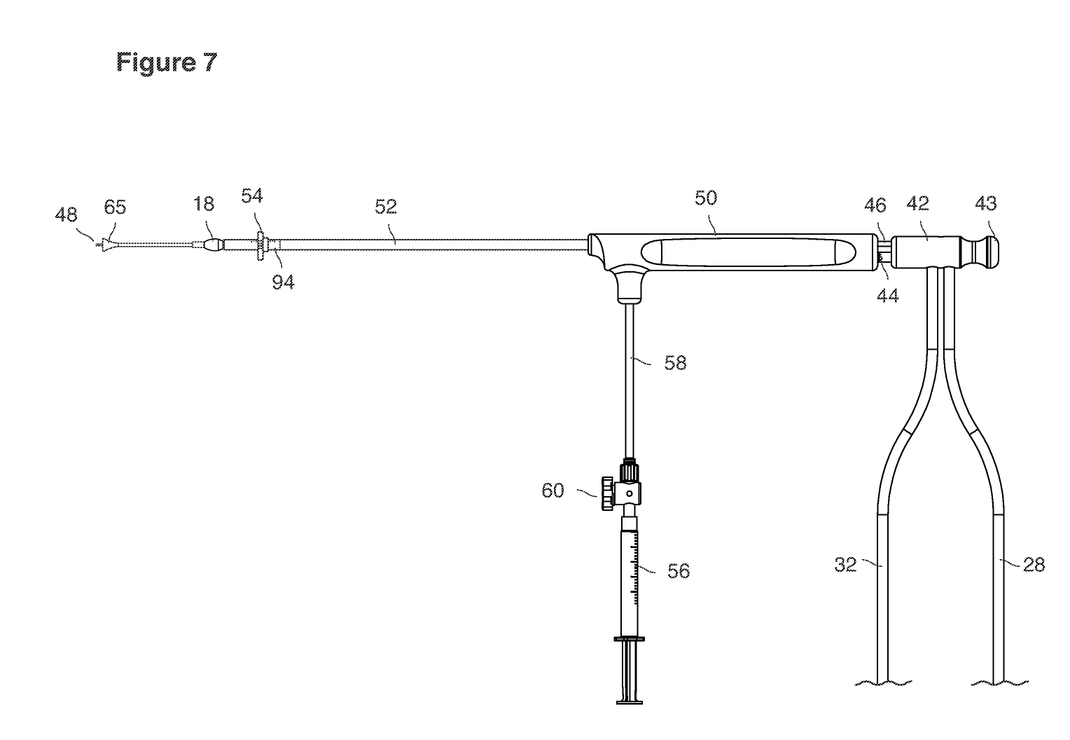

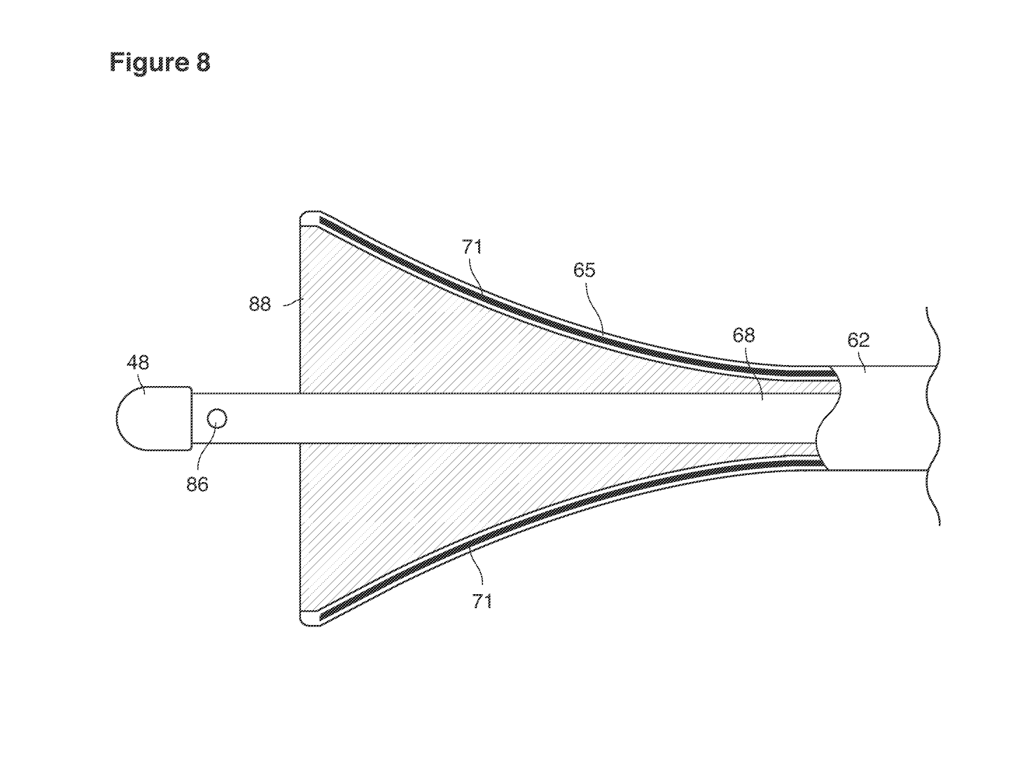

Referring to FIG. 5, the handle 50 defines a slot 80 that receives the stabilizing bar 44. When the inner catheter 40 slides axially relative to the outer guide member 38, the stabilizing bar 44 slides along the slot 80. The stabilizing bar 44 helps support the manifold 42. As illustrated in FIG. 6, the stabilizing bar 44 includes indicia 82 that indicate the extent of insertion of the inner catheter 40 relative to the outer guide member 38. The inner catheter 40 can be moved axially between the retracted position of FIG. 6, and the extended position of FIG. 7. The funnel 65, which remains compressed or folded within the outer guide member 38 when the inner catheter 40 is in the retracted position of FIG. 6, expands to its full shape when the inner catheter 40 is in the extended position of FIG. 7. The funnel 65 includes one or more funnel guides 71 (FIG. 8), which can be made from a memory-retaining material such as nitinol, that help the funnel 65 to expand and retain its shape during use. The stabilizing bar 44 terminates in a head 81 and the handle 50 includes a stop 83 which prevents the head 81 from exiting from the slot 80 such that the inner catheter 40 and the outer guide member 38 are permanently joined to form a single, integrated device, i.e., the supply/suction line 46 cannot be completely removed from the outer guide member 38 by the operator.

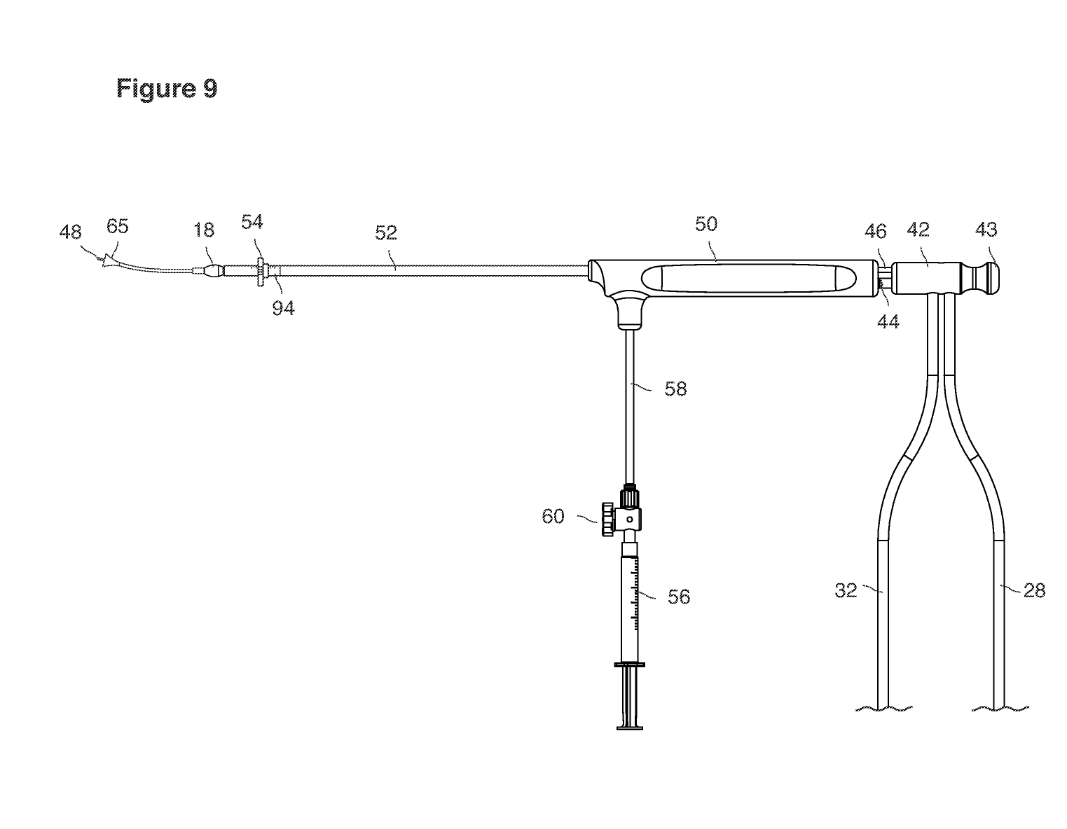

Referring to FIG. 9, the outer tubular member 62, due to the formable member 69 (FIG. 3a), can bend into a pre-shaped curve as the inner catheter 40 is extended. For example, the outer tubular member 62 can be pre-shaped according to the particular shape of a woman's uterus. Accordingly, a path that the vacuum tip 48 takes as it extends into the uterine cavity can be preset, for example, so that it follows the contoured surface of the uterine wall. Additional intrauterine manipulation of the vacuum tip 48 may be accomplished by rotating the inner catheter 40 while extending it. In some cases, the handle 50 may include a wheel to help rotate the inner catheter 40 relative to the handle 50. Alternatively, or additionally, the entire device 10 may be rotated. In some cases, the formable member 69 may include steering wires that allow the user to actively steer the vacuum tip 48.

Referring again to FIG. 5, the inner tubular member 64 of supply/suction line 62 is supported by a resin block 84 in manifold 42.

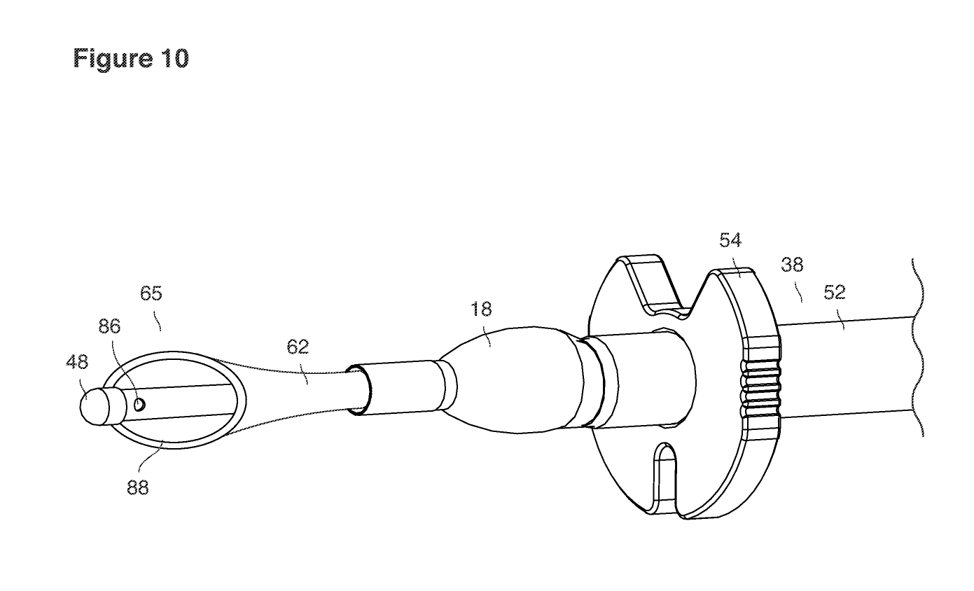

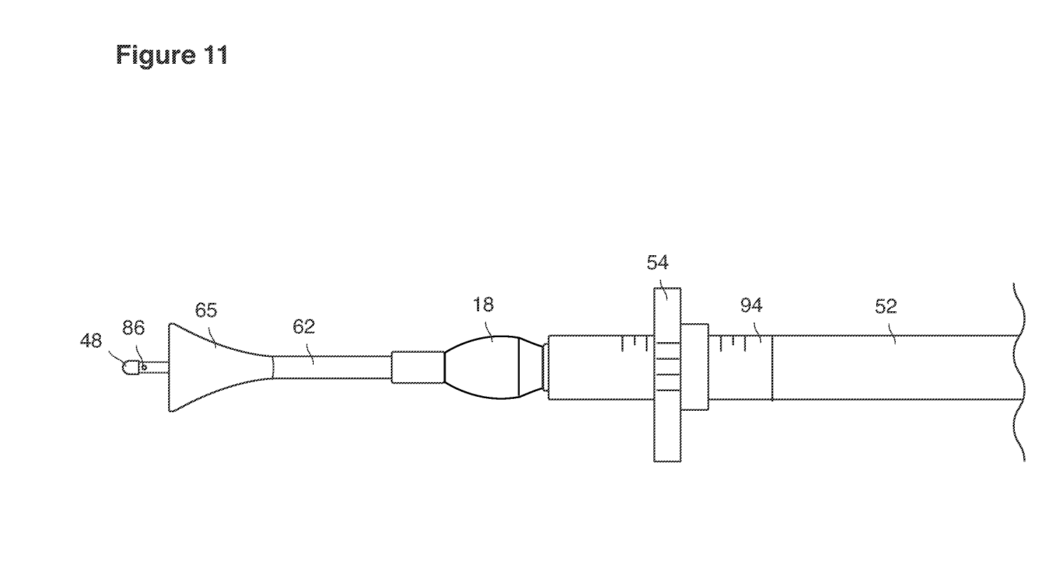

Referring to FIGS. 10 and 11, the inner tubular member 64 of the supply/suction line 46 defines a fluid supply line port 86, for example, two diametrically opposed ports, through which fluid is delivered to the uterine cavity. The ports can be circular in shape. In some implementations, the ports can be non-circular in shape to provide directional control of fluid spray. For example, the proximal side of the port can be perpendicular to the longitudinal axis of the inner tubular member 64 and the distal side of the port can diverge from the axis at an obtuse angle. The outer tubular member 62 of the supply/suction line 46 terminates at the funnel 65 positioned proximal of port 86. The funnel 65 defines a flared suction port 88 that is in fluid communication with outflow lumen 66 through which fluid and entrained blastocysts 20 are recovered from the uterine cavity. Due to the outward flare of the funnel 65, a cross-sectional area of the suction port 88 can be larger than the cross-sectional area of the outer tubular member 62. For example, the cross-sectional area of the suction port 88 can be in the range of around 1-4 mm, while the cross-sectional area of the tubular member can be in the range of around 0.5-3 mm.

As shown in FIG. 4, the lavage device 10 includes a priming cap 350 that is used to cover the ports 86 and 88 providing a seal to allow priming of the device prior to use. The position of the cervical stop 54 is adjustable relative to the balloon collar 18 along a cervical stop scale 94 (FIG. 11) on the guide arm 52. The position of the cervical stop 54 defines a dimension corresponding to a distance from an opening of the cervix at the vagina (the external cervical os) and an opening of the cervix at the uterus (the internal cervical os). The cervical stop 54 can be clamped in a set position along the guide arm 52.

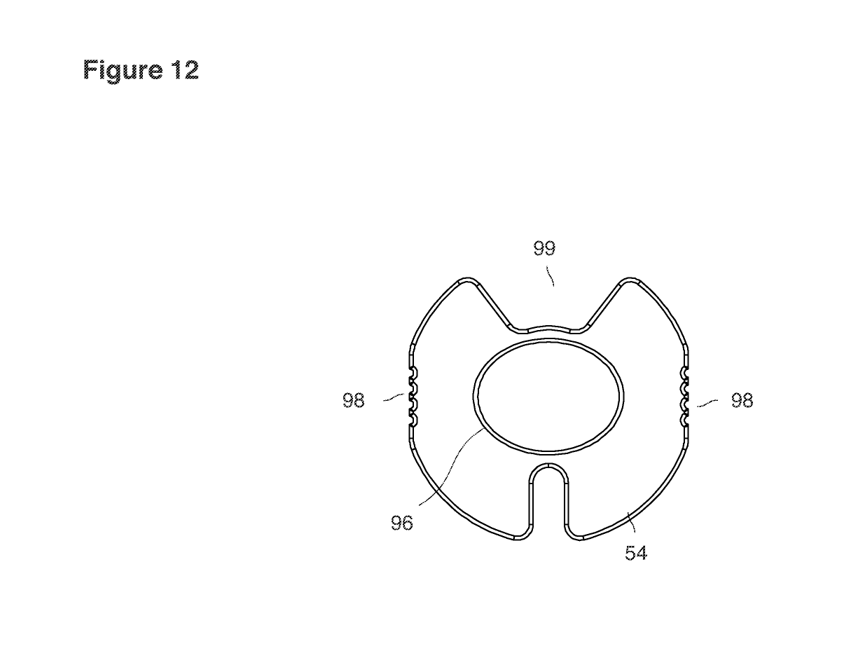

Referring to FIG. 12, the cervical stop 54 includes a locking ring 96 and flange adjustment grips 98. In its rest state, the locking ring 96 is not circular in shape and has an inner dimension smaller than the outer diameter of the guide arm 52 to lock the cervical stop 54 in position. By squeezing in on the flange adjustment grips 98, the operator can deform the shape of the locking ring 96 to a more circular shape that can slide along the guide arm 52 to adjust the position of the cervical stop 54. Upon release of the squeezing force, the locking ring 96 returns toward it rest state, locking the cervical stop 54 in place. The cervical stop 54 is shaped to have a visual port 99 that allows the operator to see the cervix and align the atraumatic tip 48 during insertion of the uterine device 10. The cervical stop scale 94 is etched into the outside of the catheter guide arm 52 and marks the position of the cervical stop when it is custom-adjusted to each patient prior to insertion.

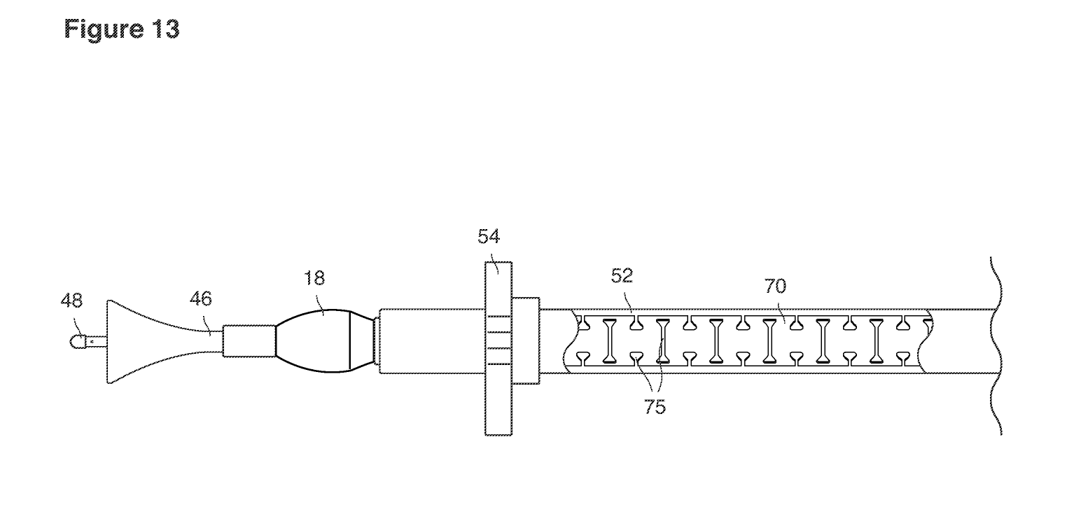

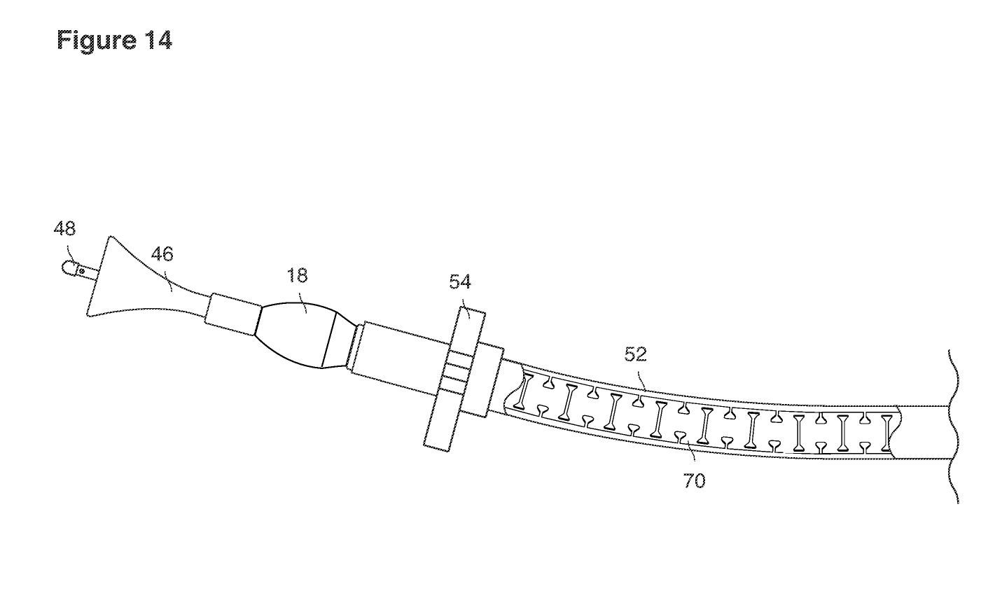

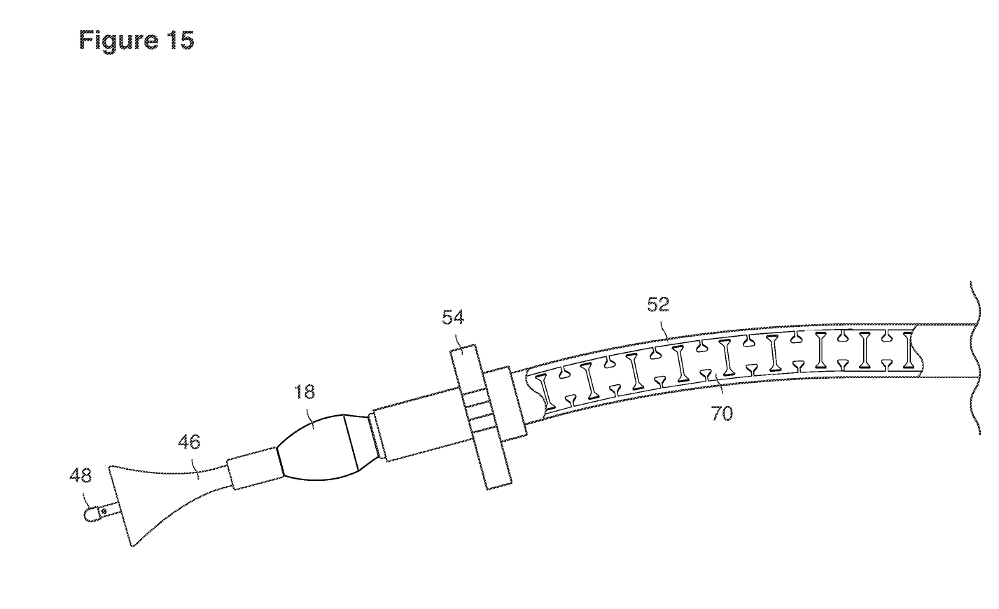

Referring to FIGS. 13-15, the formable tube 70 can be bent into a desired position by the operator to allow the atraumatic tip 48 and the supply/suction line 46 of the lavage device 10 to travel through the cervical canal and into the cervix with minimal discomfort to the patient. The angle can be preset from about 0 to 60 degrees and is customized to individual women in order to accommodate the different anatomical variations of the uterine flexion. FIG. 13 shows the formable tube 70 modified to 30 degrees up, and FIG. 14 shows the formable tube 70 modified to 30 degrees down. The formable tube 243 is made, for example, from stainless steel, is coated with polyamide, and includes cut-outs 75. The outer guide member 38 has an outer diameter in the range of, for example, 6-7 mm, and is made from, for example heat shrink polyolefin or p-bax elastomeric over layer. Inner catheter 40 has on outer diameter in the range of, for example, 3-6 mm, and for example, 3.05 mm, and is made, for example, from stainless steel. Cervical stop 54 has a diameter of, for example, 19.05 mm and is made, for example, from polyamide. The lavage device 10 is sized for use without anesthesia.

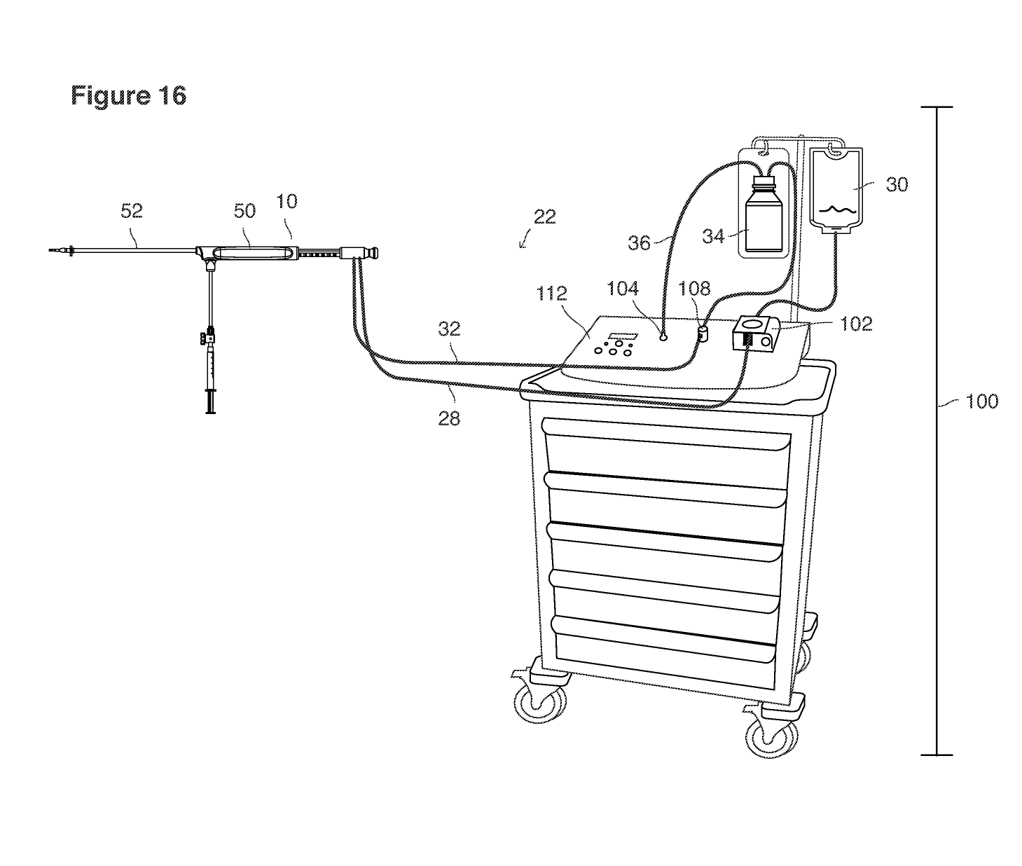

Referring to FIG. 16, the uterine lavage system 22 includes a control cart 100 used to connect the lavage device 10 to the lavage fluid bag 30 and the embryo recovery trap or collection bottle 34, and to control the inflow of fluid to the uterine cavity and the removal of fluid and entrained blastocysts from the uterine cavity. The lavage fluid bag 30 is supported by the cart 100, and the supply line 28 is routed from the fluid bag 30 through a peristaltic fluid pump 102 to the lavage device 10. Blastocysts 20 are recovered through the lavage device 10 and travel to the collection bottle 34 via the suction recovery channel 32. The collection bottle 34 is connected to a vacuum supply connector 104 via the suction line 36 through which suction is applied to suction recovery channel 32. The application and level of suction is controlled by a pinch valve 108. The introduction of fluid is controlled by the pump 102. The lavage fluid is drawn from the bag 30, pumped through the supply line 28, and pulsed in and out of the uterus through the atraumatic tip 48. The pump 102 supplies uterine lavage fluid in a pulse rhythm with a vacuum element that alternates suction and pulses cadenced the opposite to the fluid delivery at a preset frequency of, for example, 0.5 to 4.0 seconds with less fluid being aspirated than delivered to ensure that air is not introduced into the uterine cavity.

The control system manages pulse and flow via electro-mechanical means (software instructs the control system in use of vacuum and pulse of fluid delivery). The control system is reprogrammable such that software can be loaded that alters the pulse frequency, the pressure of fluid supply, the frequency of vacuum pressure, amount of vacuum supplied, and the frequency and duration of pause steps between pressure and vacuum supply.

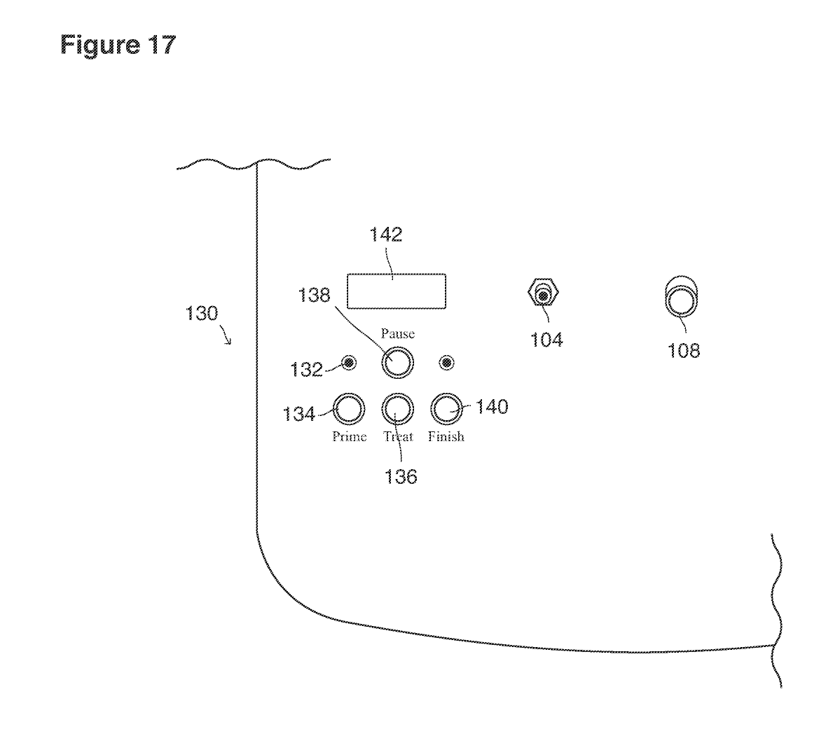

Referring to FIG. 17, a user interface 130 for controlling the system 22 includes a power button 132, a prime button 134, a treat button 136, a pause button 138, and a finish button 140. The power on/off button turns on an electrical power supply to the control system. The Prime button starts the fluid supply pump and keeps the pump running for the duration of the time that the button is depressed. The Treat Button starts the lavage cycle invoking the software to execute a pattern of pulse-pause-vacuum-pause until the fluid supply is utilized fully. The Finish button stops the lavage cycle. Faults in the set-up of the lavage device or with the software during the lavage cycle are indicated on a LED screen 142 and the control system automatically pauses the lavage cycle until the problem is resolved. The user interface 130 produces a series of electronic beeps indicating when a portion of the lavage cycle is completed. Beeps occur after each treat cycle and after the finish cycle is completed.

The IV bag 30 is a standard format, latex free, PVP free, DEHP free IV bag that can hold requisite lavage fluid solutions. The IV bag holds no more than the total amount of lavage fluid to be used in the lavage cycle. The IV bag is attached to the lavage system via a standard spike and tube format. The IV bag is translucent such that the operator can monitor fluid movement from the IV bag though the tubing and the catheter.

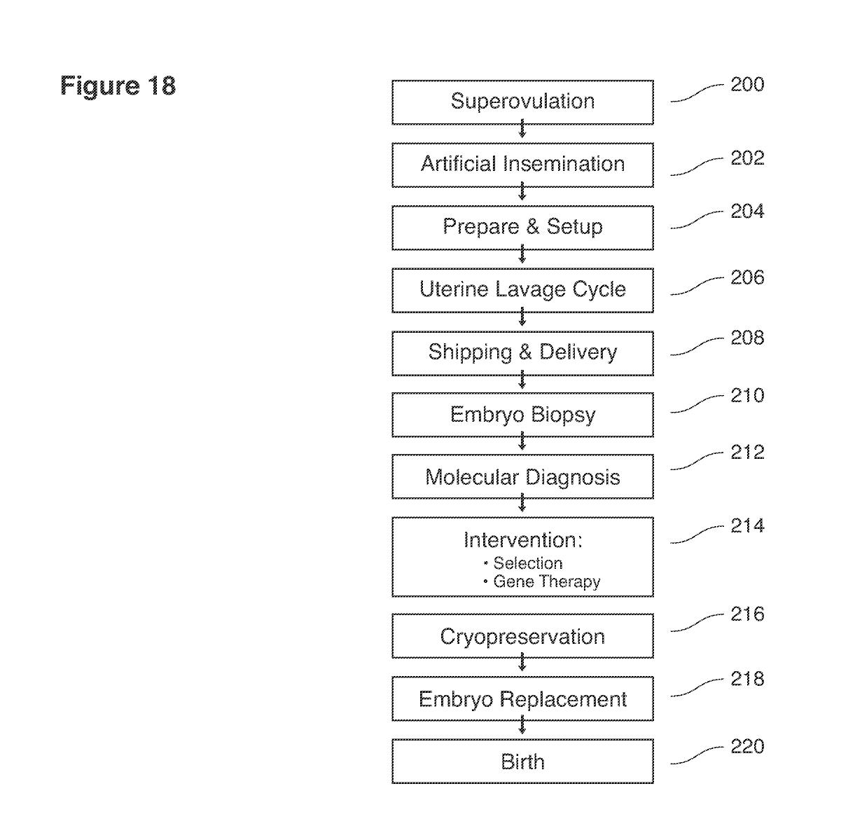

Referring to FIG. 18, the lavage system 22 is used in one or more steps of a procedure that includes superovulation 200, artificial insemination 202, preparation and set up 204, uterine lavage cycle 206, shipment and delivery of blastocysts recovered during the lavage process 208, shutdown cycle 222, embryo biopsy 210, molecular diagnosis 212, intervention 214, cryopreservation 216, embryo replacement 218, and ending in the birth 220 of a healthy baby.

Preparatory to lavage, prior to superovulation and insemination, a practice lavage can be performed (approximately one or two months) before the live procedure is scheduled. In the practice lavage, measurements are taken (with the assistance of imaging technologies) and the lavage device 10 is custom fit to enable the anatomy of each patient to be accommodated. Precise imaging of each woman's anatomy utilizes imaging devices, for example, two-dimensional or three-dimensional ultrasound, magnetic resonance imaging, or other imaging technology. The operator determines the optimal position for cervical stop 54 and records the reading on the scale 94, the optimal insertion of stabilizing bar 44 and records the reading on the indicia 82, the angle the lavage device is to be set at by modification of the formable tube 70, and the amount of inflation of the balloon collar 18 to accommodate the degree of cervical dilation of the patient.

Superovulation is caused in a woman to form multiple corpora lutea that undergo apoptosis and cannot support development of a viable implanted pregnancy following shutdown 222. In-vivo fertilization of multiple oocytes by artificial insemination and/or natural insemination is followed by maturation of the fertilized oocytes to form multiple mature preimplantation embryos that present to the uterine cavity as blastocysts.

To cause superovulation, FSH is delivered to the woman's body. The FSH can be delivered by self-injection. The dosage of FSH is appropriate for induction of superovulation, in vivo fertilization, and embryonic maturation. The FSH is, for example, self-injected daily for 5 to 15 days in the range of 5 to 600 mlU per day. The FSH includes at least one of injectable menotropins containing both FSH and LH; purified FSH given as urofollitropins; recombinant pure FSH; or single doses of long acting pure FSH (recombinant depot FSH), including administering GnRH antagonists to quiet the ovaries while causing superovulation. The GnRH antagonists include receptor blocker peptides. The GnRH antagonists include at least one of Cetrotide 0.25 to 3.0 mg, Ganirelix, Abarelix, Cetrorelix, or Degarelix in which causing superovulation includes administering GnRH including administering a single dose of hcG agonist subcutaneously or snuffed to trigger the superovulation. The GnRH includes at least one of Leuprorelin, Leuprolide acetate, Nafarelin, or Naferelin acetate snuff 117 including administering LH or hCG without GnRH agonist including administering LH or hCG or in combination with GnRH agonist in which impaired (apoptosis) corpus luteum estradiol and progesterone production is supplemented to maintain embryonic viability and maturation by including administrating progesterone and estradiol until recovery of the blastocysts. The progesterone includes at least one of vaginal progesterone, or oral progesterone and the estradiol includes at least one of oral or transdermal estradiol. The progesterone includes Crinone® 1 application per day or Prometrium 200 mg® 3 applications per day or Prometrium 200 mg® 3 oral capsules per day, and the estradiol includes transdermal estradiol patches 400 ug per day or oral estradiol 0.5 to 5.0 mg per day in which blastocyst implantation is prevented by discontinuing administration of estradiol and progesterone starting on the day of blastocysts recovery on the day of lavage. Desynchronization includes administering progesterone receptor antagonist. The administering includes a single dose of progesterone receptor antagonist (Mifepristone 600 mg) injected into the uterine cavity with a second dose (Mifepristone 600 mg) mg given by mouth one day prior to expected menses. Desynchronization includes administering GnRH antagonist on the day on which the blastocysts are recovered to induce further corpus luteum apoptosis, suppress luteal phase progesterone, and further decrease risk of a retained (on account of blastocysts missed by the intrauterine lavage) pregnancy. The GnRh antagonist includes Cetrotide 0.25 to 3.0 mg.

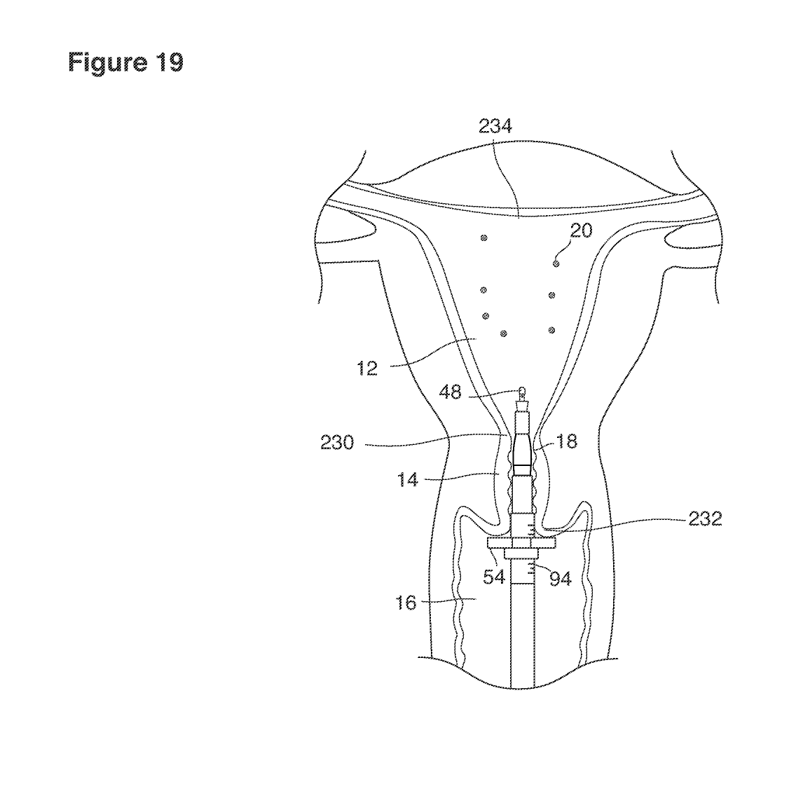

Uterine lavage is typically performed between 4 and 8 days after the LH dose or LH surrogate trigger that released in vivo the multiple oocytes resulting from the superovulation. Referring to FIG. 19, at the optimal time (most likely day 6), the blastocysts 20 are located between the anterior and posterior uterine walls at approximately the geometric center of the uterine cavity 12. This location is in close proximity to the ultimate site of implantation, which is believed would take place within one day or less after the procedure if the blastocysts 20 were not recovered.

In preparation for the live lavage, the disposable and reusable elements of the instrument are selected based on the prior measurements and study of the woman's anatomy, and assembled and attached to the pulsing and suction elements, ready for the procedure. The operator sets the cervical stop 54 at the position determined on the cannula that ensures the balloon collar 18 is positioned along the internal cervical os 230. The cervical stop 54 is set relative to the measurement markings on the cervical stop scale 94 that defines the distance from the balloon collar 18, which has been premeasured by the device operator, and is clamped to the catheter guide arm 52.

The operator then shapes the catheter guide arm 52 as predetermined by the operator such that when the lavage device 10 is placed into the uterus the atraumatic tip 48 is positioned for extension along the midline of the uterus. The catheter guide arm 52 is flexible and will hold its shape via internal formable tube 70, and is bent into position to accommodate the position of the uterus relative to the particular woman's body (anteverted, retroverted, cast medially or laterally or any combination therein). The anatomy of the patient in question has been documented in prior exams such that the uterus position information can be used to prepare the lavage device for the uterine lavage cycle.

Temperature preparations are completed such that prior to the lavage cycle the fluid bag 30 with lavage fluid is pre-heated to 37 degrees Celsius by placing the fluid bag on a heating plate for a period of 30 minutes. The embryo recovery trap 34 is preheated for 30 minutes by placing a heating wrap around the container. This step ensures that the blastocysts 20 will be sustained at 37C for the time period just after removal from the uterus through the arrival at an embryology laboratory.

Prior to the lavage cycle, the operator primes the lavage device 10 with lavage fluid as follows: turns on the lavage device controller by pressing the ‘Power’ button 132 (FIG. 17) located on the control panel of the controller; presses and holds the ‘Prime’ button 134 on the control panel of the controller; and holds the ‘Prime’ button 134 down until the lavage fluid is pumped through the fluid supply line 28 and the suction recovery channel 32 of the lavage device 10 and deposits fluid into the embryo recovery trap 34. After priming is complete, the operator removes the priming cap 350 and the device is ready for insertion into the patient.

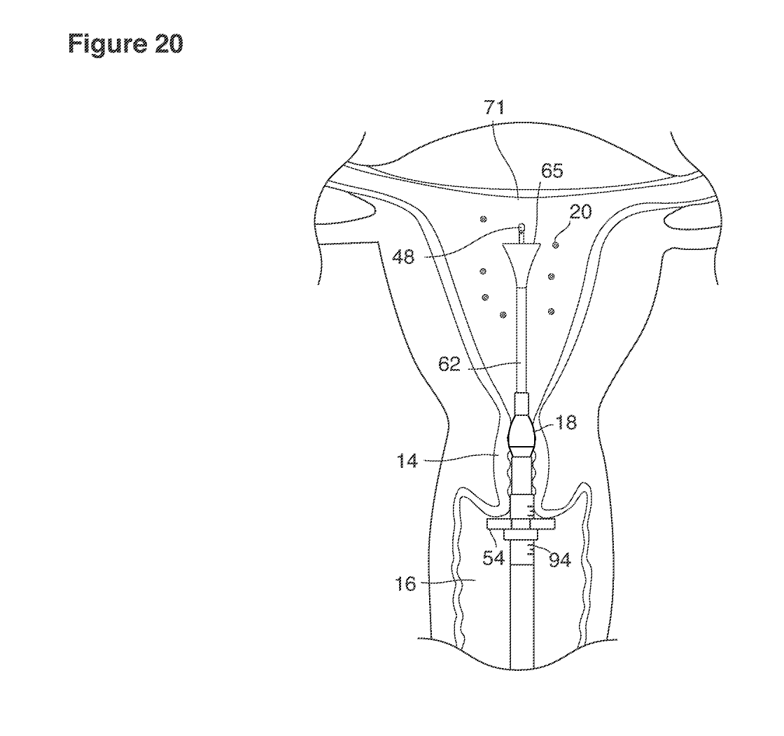

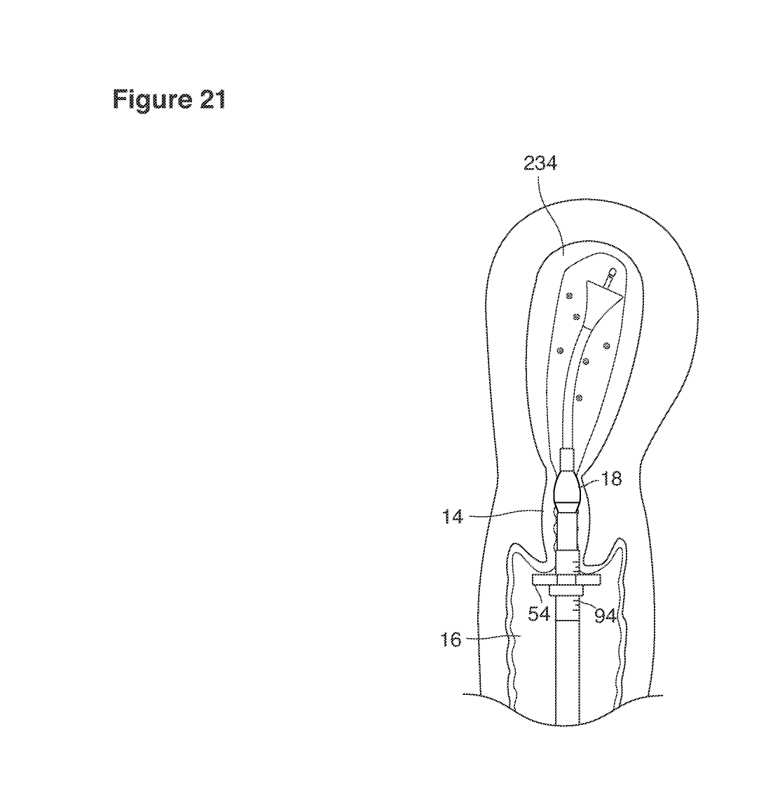

The lavage procedure is conducted as follows:
i) Intracervical Insertion: The procedure begins with insertion of the lavage device 10 into the uterine cavity 12 via the cervical canal 14 through the vagina 16. The lavage device 10 is inserted until the cervical stop 54 rests against the external surface of the cervix 14 (external cervical os 232) creating a fluid-tight seal, protecting the vagina 16 (FIG. 19). The deflated balloon collar 18 lies at the end of the cervical canal 14 at the entrance to the uterus (internal cervical os 230).
ii) Insufflation: Creation of Cervical Seal: The cervical seal balloon collar 18 is then inflated (FIG. 1) to provide a watertight seal at the internal cervical os 230 to prevent the loss of lavage fluid around the lavage device 10. This is done by depressing the syringe 56 until 1.5 cc to 3 cc of fluid, air or liquid, is injected into the balloon collar 18, or until sufficient resistance to balloon inflation is felt by the operator. The stopcock 60 is then closed to ensure the balloon collar 18 remains inflated throughout the duration of the procedure. In some cases, especially for nulliparous women, balloon inflation may not be required to gain a seal at the internal cervical os 230.
iii) Positioning of Catheter Tip on Inner Surface of Uterus: The final step prior to performing the lavage cycle is positioning of the vacuum tip 48 on or close to the inner surface of the uterine cavity 12. The operator utilizes predetermined dimension information that specifies the length of the uterus from the external cervical os 232 to the fundus 234 to set the position of the catheter tip 48 as follows: hold the lavage device using the handle 50; extend the atraumatic tip 48 into the uterine cavity 12 (FIGS. 20 and 21) by pushing the manifold 42 slowly forward until the tip 48 and/or the funnel 65 touches the inner wall of the uterine cavity 12. The operator knows when contact with the inner wall has been made when resistance is felt as the outer tubular member 62 is being extended into the uterus while depressing the manifold 42. The lavage device 10 including its fluid supply and vacuum lines is now in its extended position. By positioning the vacuum tip 48 and/or the funnel 65 at the surface of the wall of the uterus, the lavage device 10 can utilize mechanical agitation, that is touching and agitating the surface, to help loosen mucus and recover blastocysts in the mucus.

Alternatively, the position of the atraumatic tip 48 is determined by monitoring the indicia 82 on the stabilizing bar 44. By further pre-bending the outer tube member 62 to a desired shape, the path of the vacuum tip 48 within the uterine cavity 12 can be estimated.

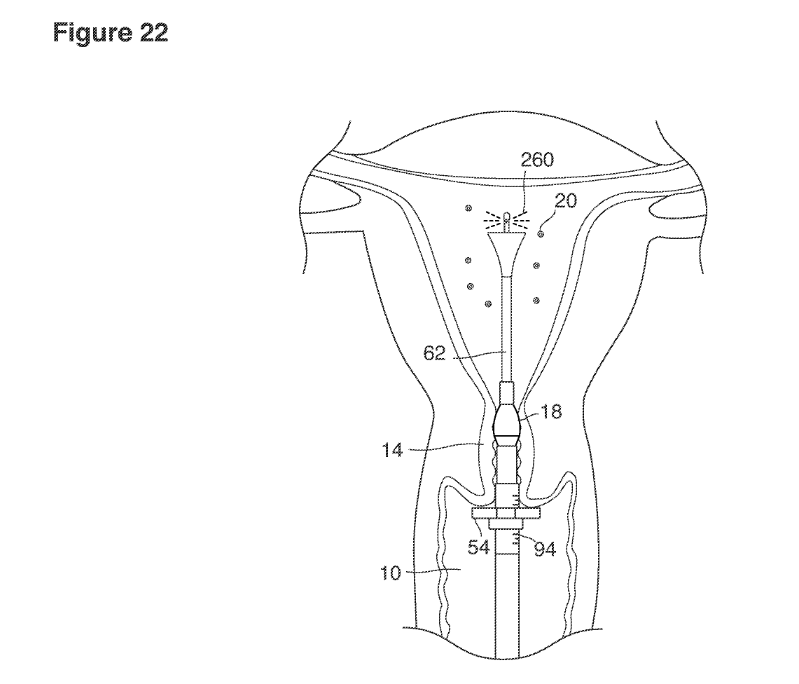

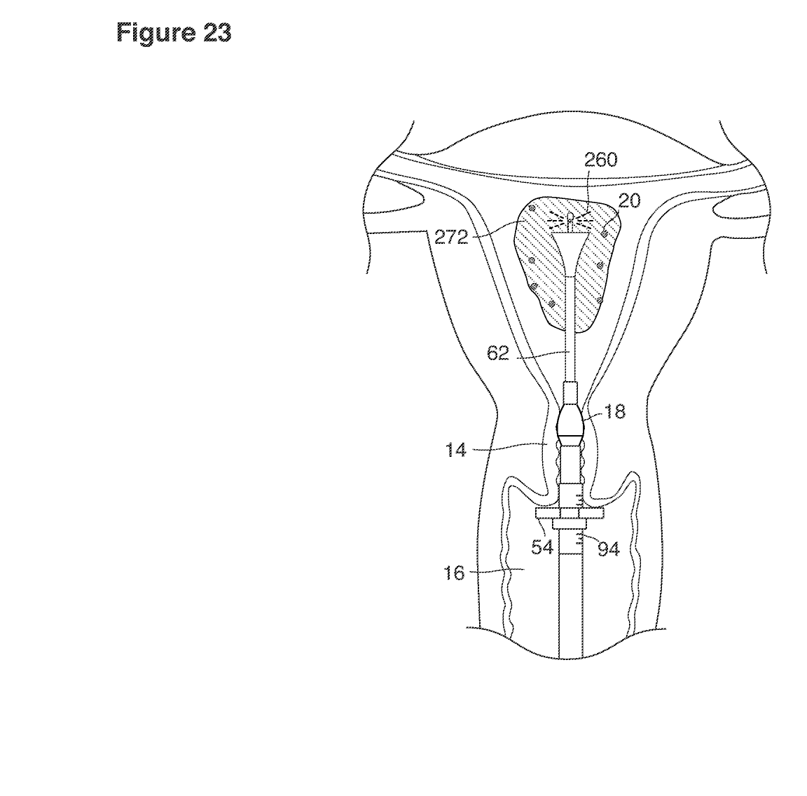

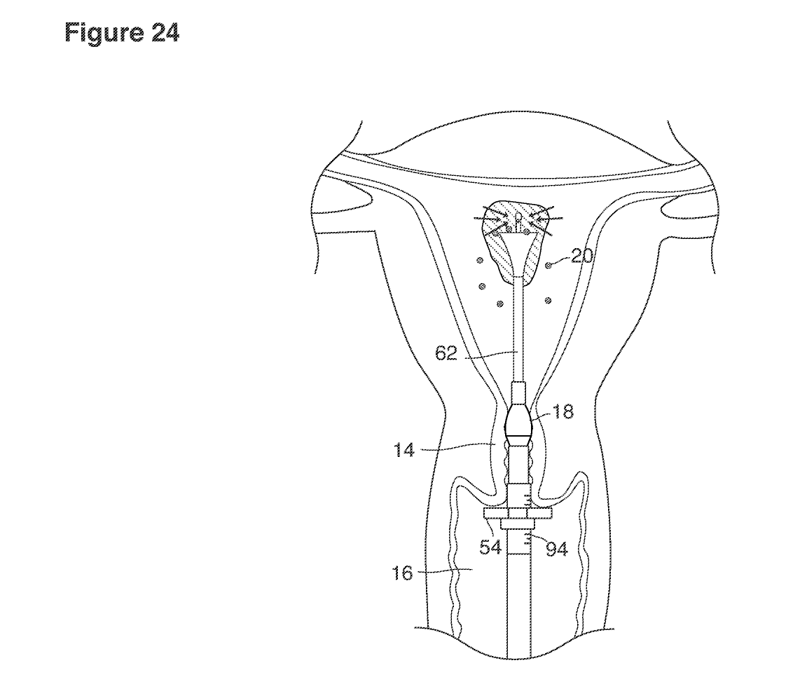

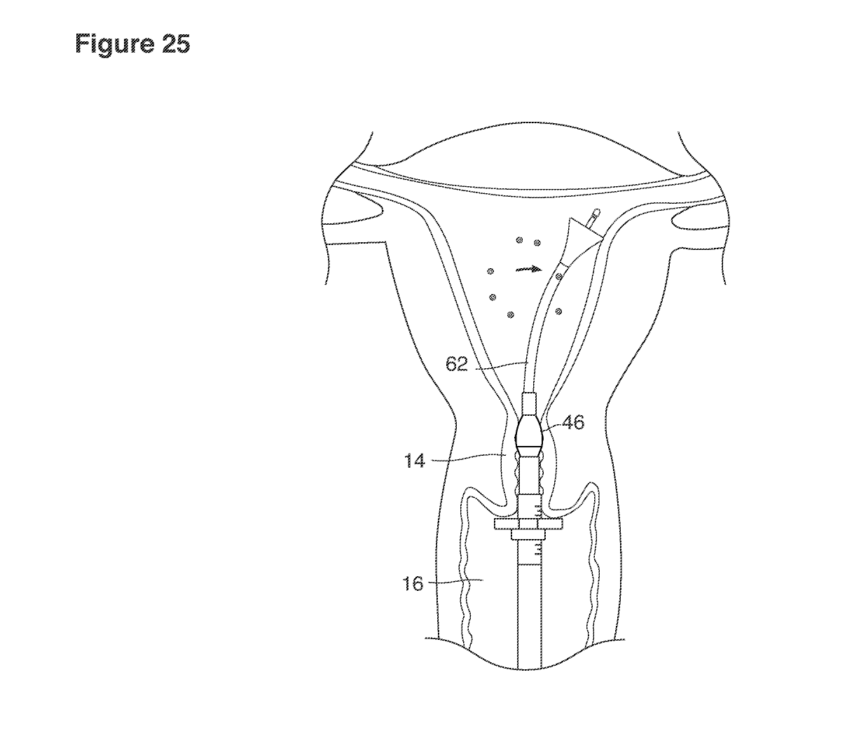

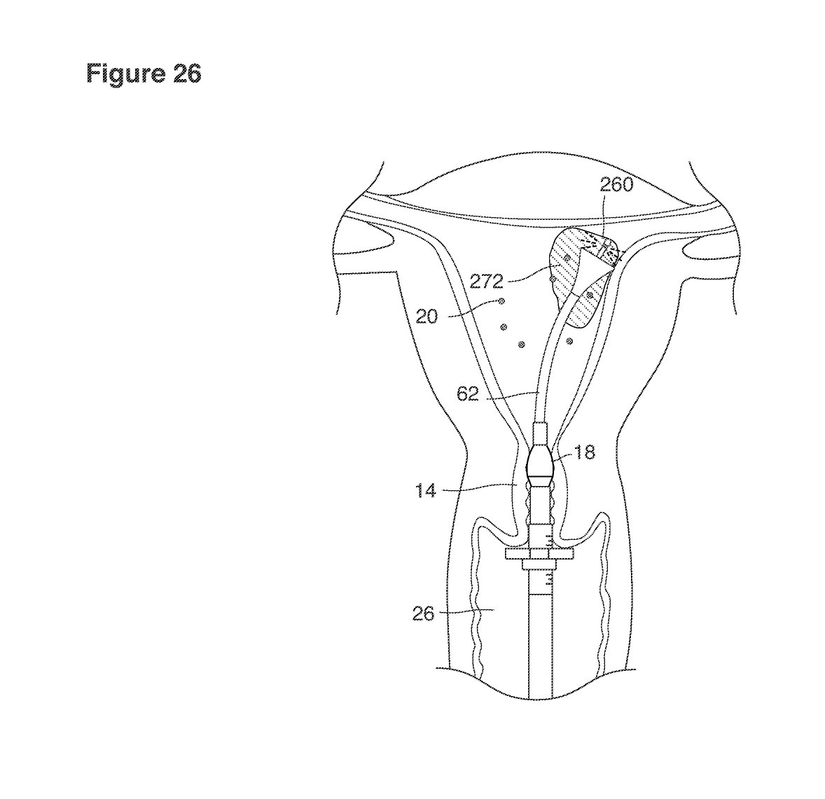

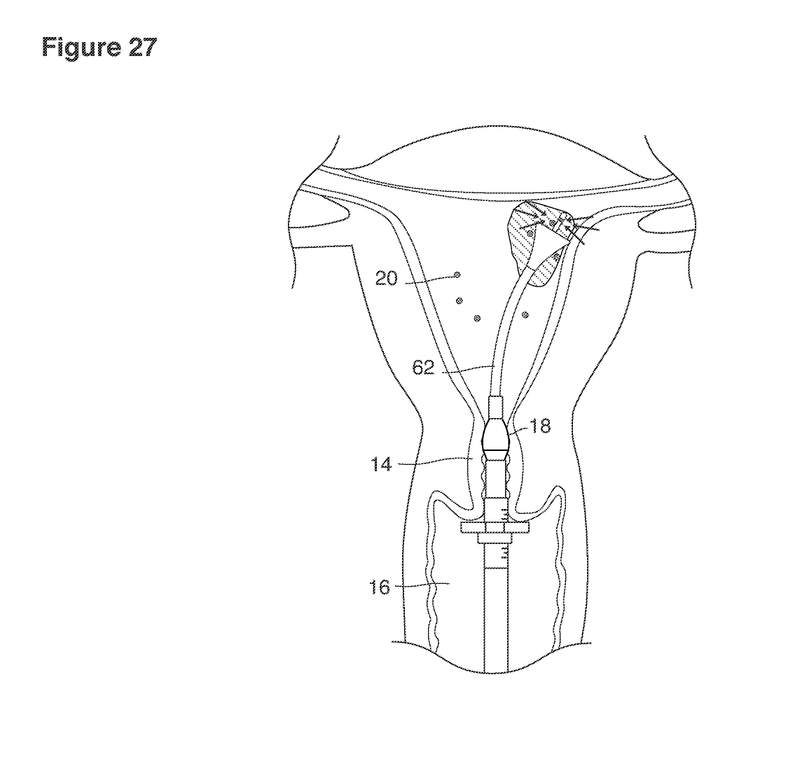

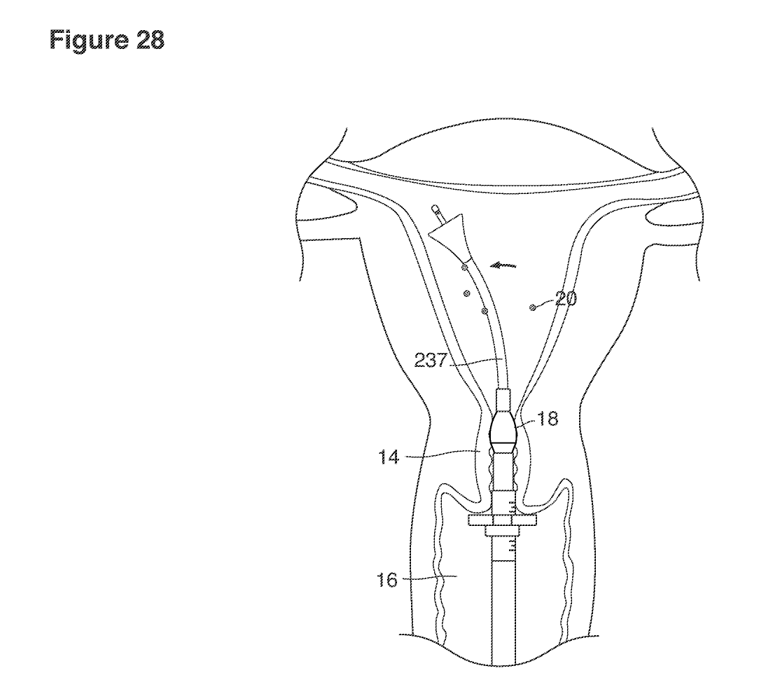

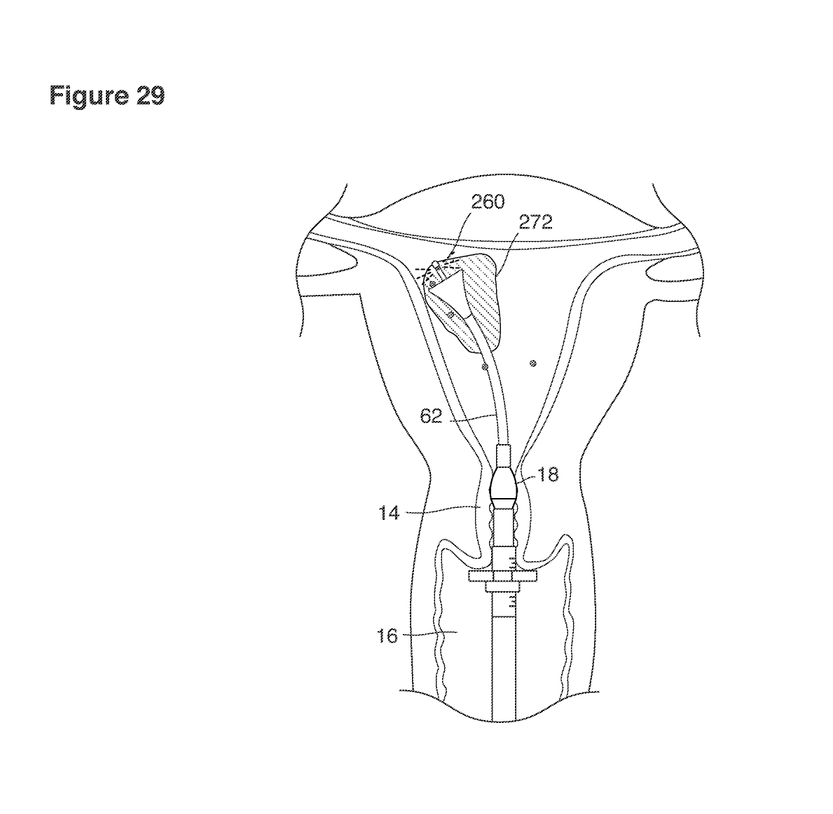

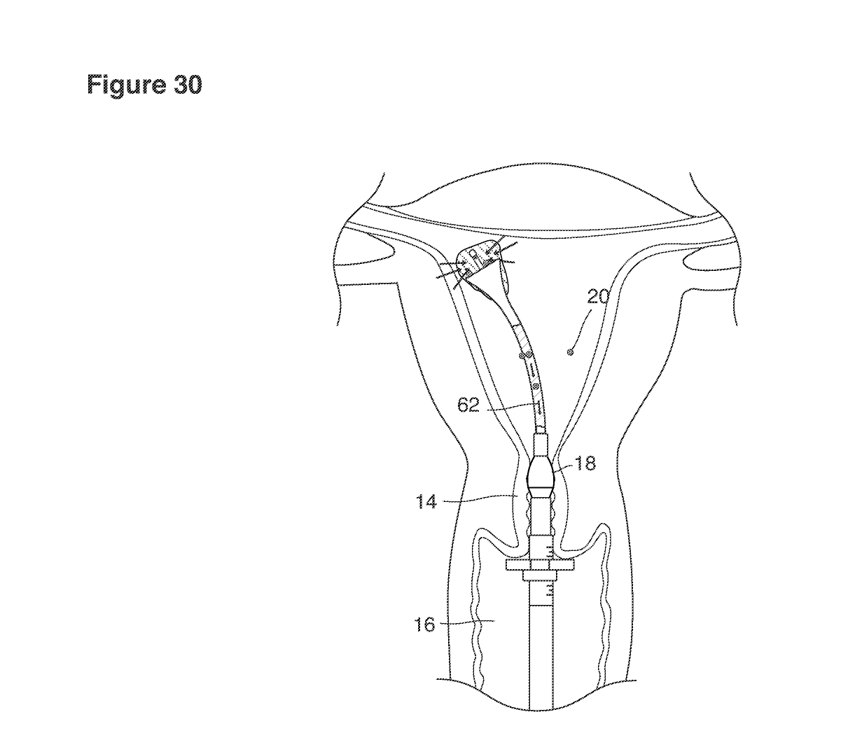

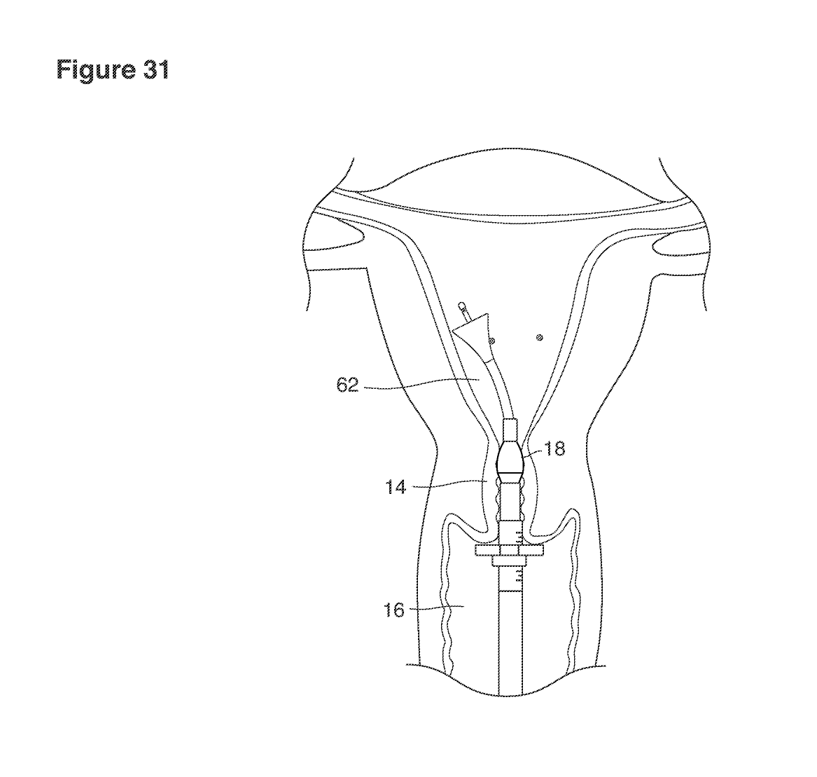

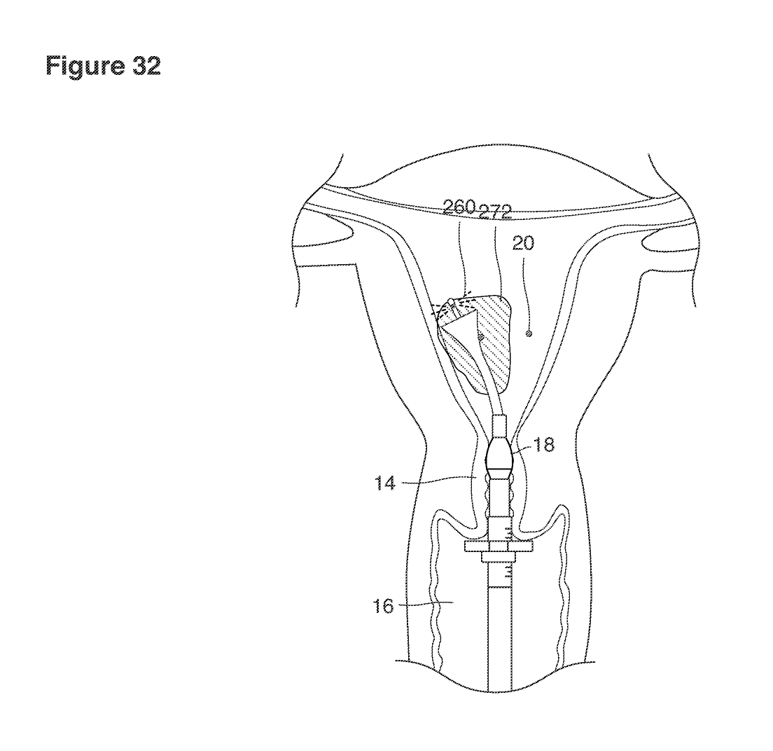

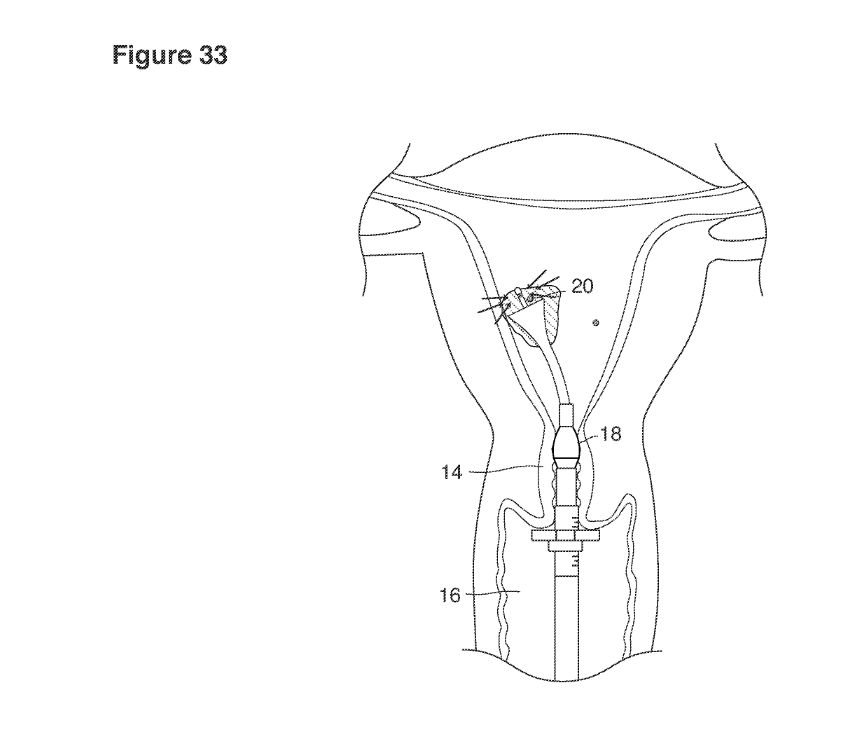

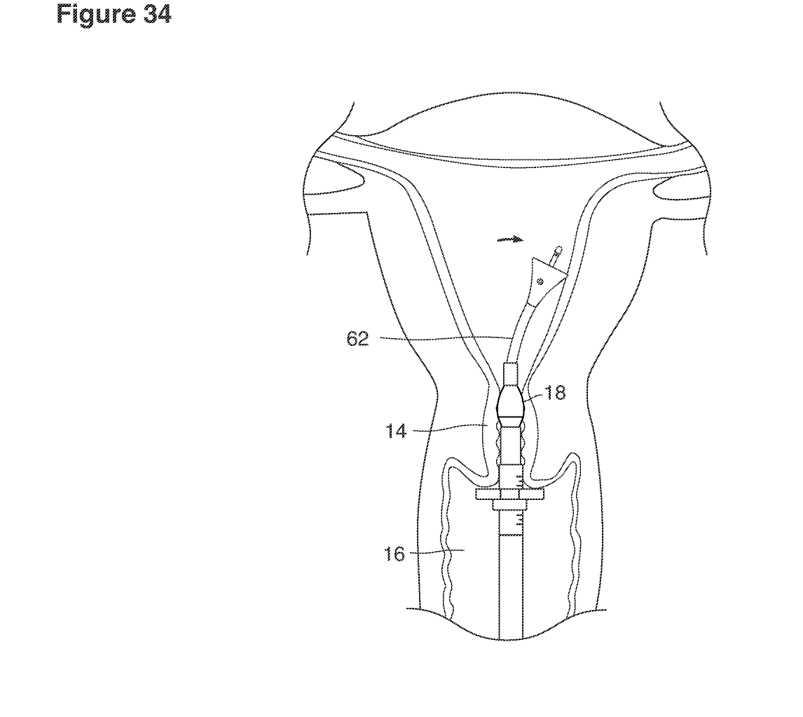

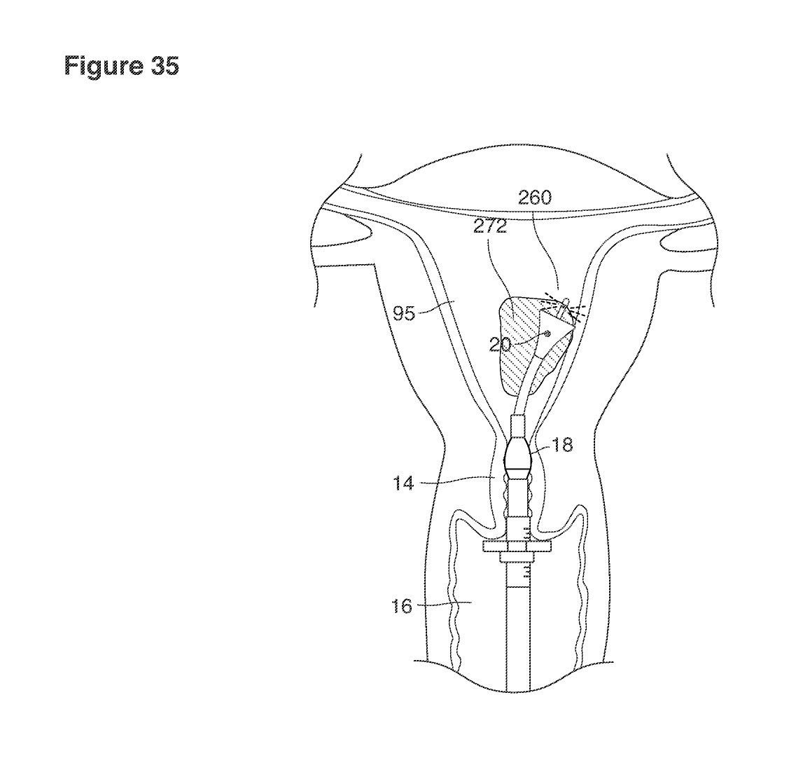

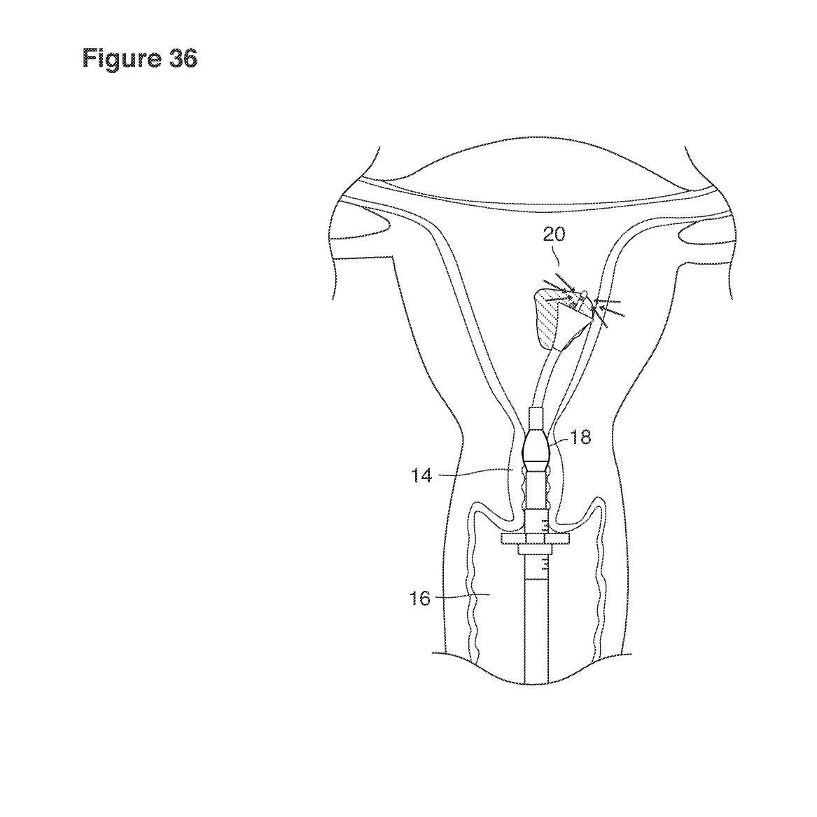

iv) Uterine Lavage & Embryo Recovery: The lavage cycle (FIGS. 22-36) is started by depressing the ‘treat’ button on the control panel. The first stage of the lavage cycle is begun by injecting a small amount of fluid 260 (FIG. 22) into the uterine cavity 12 for form a puddle 262 of fluid (FIG. 23) encompassing the blastocysts 20. All of the fluid present in the uterine cavity 12 is then suctioned into the suction port 88 of the funnel 65 (FIG. 24) along with one or more entrained blastocysts 20. Subsequent stages of the lavage cycle are begun by manipulating the vacuum tip 48 along the wall of the uterus by, for example, rotating, extending, and/or retracting the outer tubular member 62 (FIGS. 25, 28, 31, and 34). This process can cause further mechanical agitation of the uterine wall to help loosen the mucus. After repositioning the vacuum tip 48, injection of the fluid 260 to form the puddle 262 (FIGS. 26, 29, 32, and 35), and suctioning the fluid (FIGS. 27, 30, 33, and 36) encompassing the blastocysts into the suction port 88 can be performed as described above.

The lavage cycle is repeated and controlled by the lavage device controller. The lavage cycle operates for approximately 3 minutes, or until 100% of the lavage fluid (maximum 5 minutes) located in the fluid bag 30 is cycled through the lavage device 10, into the uterus and removed via the suction recovery channel 32 into the embryo recovery trap 34. The operator monitors the lavage cycle visually by watching fluid flow. While the lavage cycle is operating the fluid flow will pulse through the fluid supply line 28 and suction recovery channel 32. The fluid quantity will decrease in the fluid bag 30 and increase in the embryo recovery trap 34. The recovered lavage fluid will appear cloudy due to presence of uterine fluid and endometrial tissue captured from the lavage process and recovered from the uterus. The embryos are withdrawn from the uterus with an efficiency of at least 80%. The embryos are withdrawn from the uterus with an efficiency of at least 90%. The embryos are withdrawn from the uterus with an efficiency of at least 95%. Desynchronization of the endometrium is caused to reduce the chance that any embryos remaining in the uterus will form a viable pregnancy.

v) Jamming: (optional step to address lack of fluid flow in catheter during the lavage cycle): Jamming is the term which describes a lack of fluid flow and can occur due to the buildup of endometrial tissue at the atraumatic tip 48. The following steps can be taken in the event of jamming: press the Pause button on the lavage device controller control panel, adjust the position of the catheter tip and restart the lavage cycle, repeat as needed, when flow is detected in the suction recovery channel allow the lavage cycle to complete.

vi) Completion and Stop of the Lavage Cycle: The lavage cycle is complete when (1) the fluid bag is empty and (2) the controller system has operated for at least one minute after all fluid is visibly removed from the fluid bag, supply line and suction recovery channel. The lavage procedure automatically ends after a sustained duration of vacuum only cycle is completed or when the operator depresses the ‘Finish’ button twice. The operator then turns off the lavage controller by depressing the power button.

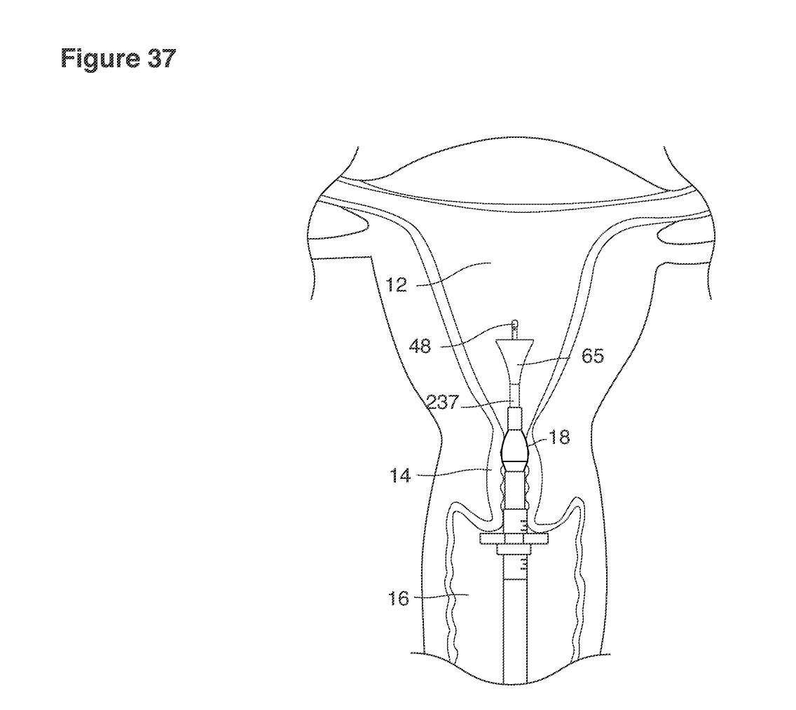

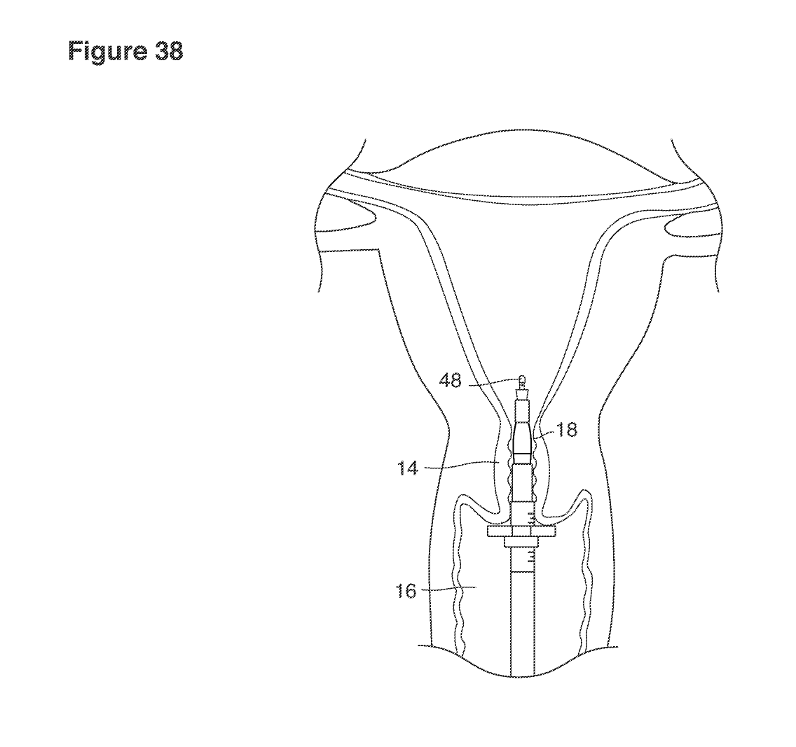

vii) Removal of Lavage device: The operator removes the lavage device as follows: pull the manifold 42 away from the handle 50 to retract the inner catheter 40 into the outer guide member 38 (FIG. 37); deflate the balloon collar 18 by opening the stopcock 60 and retracting the syringe 56 to 0 cc (FIG. 38); the lavage device 10 is then slowly removed from the cervix 14.

The fluid used in the lavage cycle may be lactated Ringers, HTF (Human Tubal Fluid), modified HTF, or HEPES-buffered media. The operator determines appropriate solutions based upon knowledge and preference. The operator receives recommendations as follows for fluid choice: (1) non-heparin based media (2) non CO2 based media that is approved/generally accepted for use in humans.

The uterine lavage procedure is performed under low flow and vacuum conditions, not to exceed the maximum pressure allowed by the device of between 2 ounces per square inch and 20 pounds of pressure per square inch and 10-14 Hg of vacuum pressure to maintain the integrity of the blastocysts during fluid delivery and removal. The uterine cavity is not expanded or pressurized. The lavage device 10 does not include any members that act to expand the uterine cavity, as such an expansion can introduce air into the uterine cavity, which can kill the blastocysts 20. The lavage process, as well as its preparatory steps and finish instructions, are designed to prevent the introduction of air into the uterine cavity to ensure the health and integrity of the recovered blastocysts.

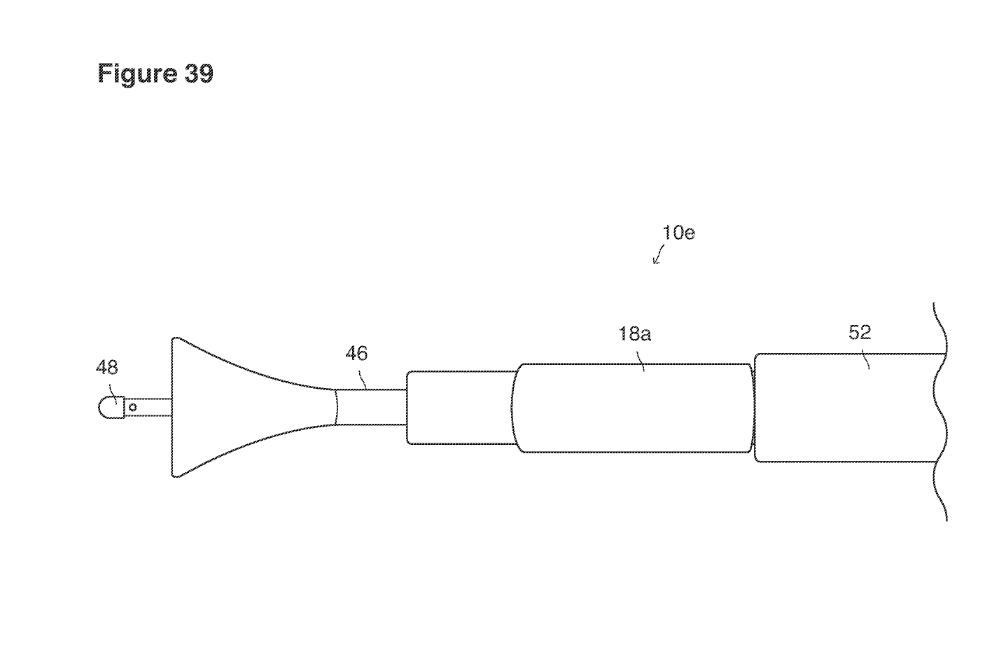

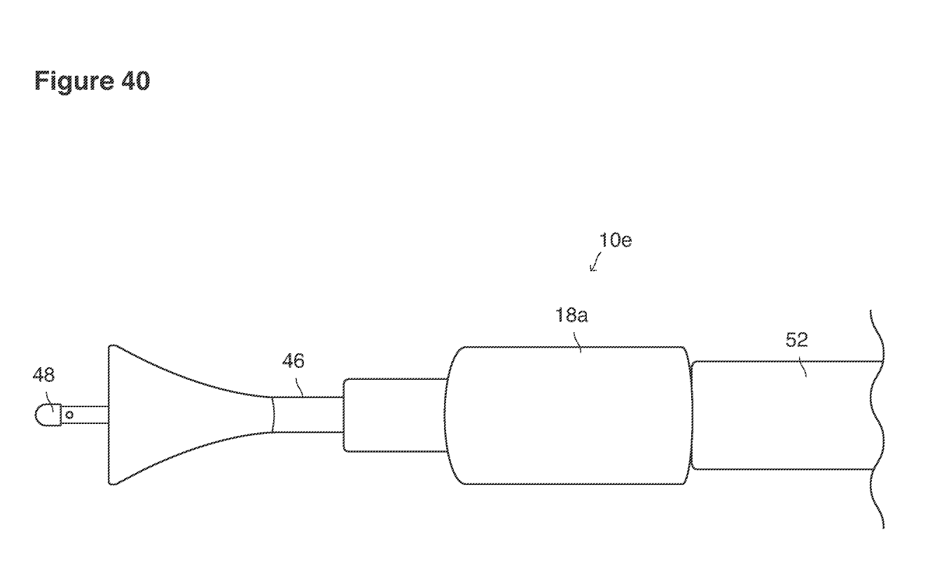

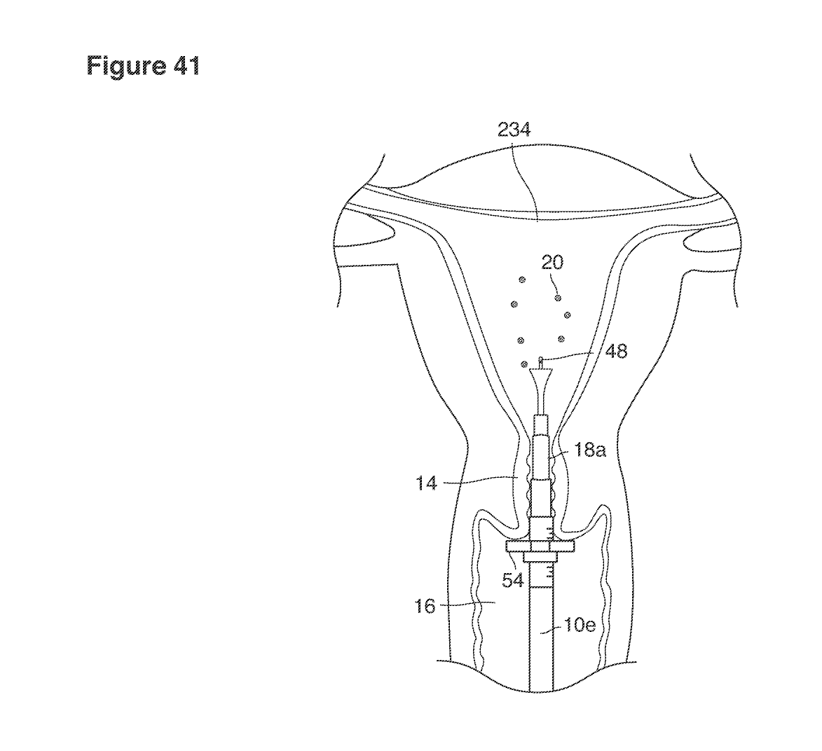

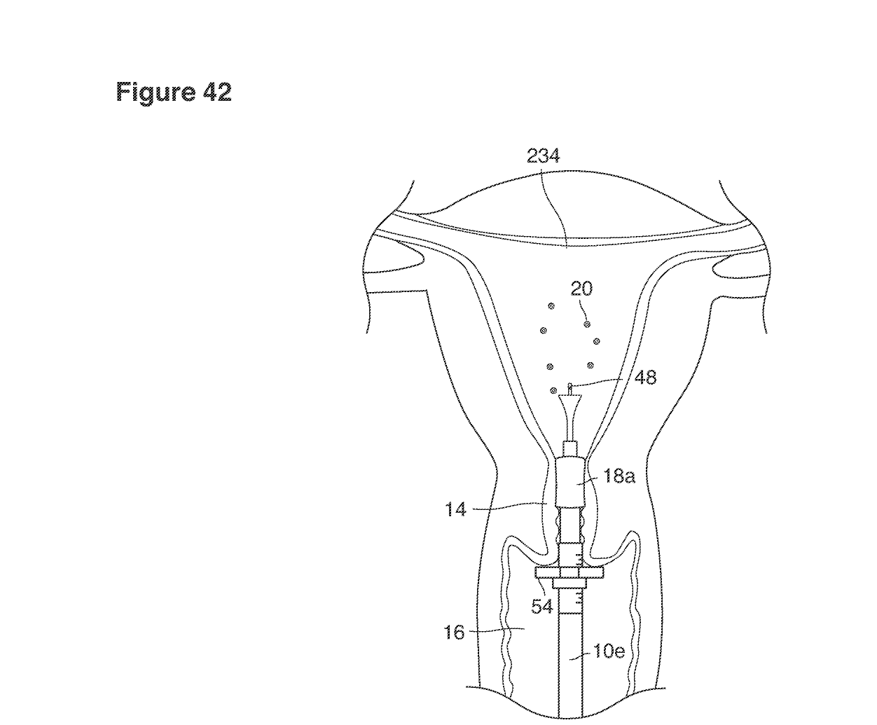

Referring to FIGS. 39-42, a lavage device 10e includes an activatable seal in the form of expandable foam 18a. The foam 18a is compressed prior to insertion and expands within the cervix to seal the uterine cavity from the external environment, as illustrated in FIG. 51.

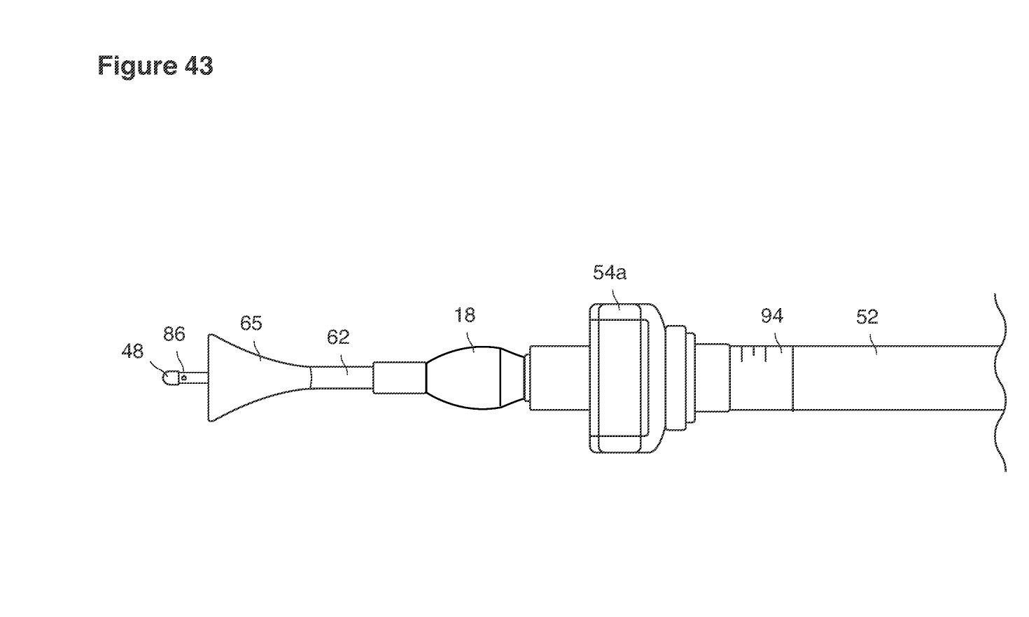

In some implementations, the cervical stop 54 can be replaced with a cervical cup 54a (FIG. 43). The position of the cervical cup 54a can be adjustable relative to the balloon collar 18 along the cervical stop scale 94 on the guide arm 52. The position of the cervical cup 54a defines a dimension corresponding to a distance from an opening of the cervix at the vagina (the external cervical os) and an opening of the cervix at the uterus (the internal cervical os). The position of the cervical cup 54a may be fixed in position relative to the guide arm 52 prior to insertion of the device 10. The cervical cup 54a can be made from a flexible material, such as polyamide, and can have inner and outer diameters in the ranges of, for example, 3-9 mm and 6-12 mm, respectively. In some cases, the cervical cup 54a may be fixedly attached to a distal end of the guide arm 52. In this case, the relative position of the cervical cup 54a to the balloon collar 18 may be adjusted by extending and retracting the support member 74 relative to the guide arm 52.

In some implementations, vacuum may be applied to the cervical cup 54a to attach and seal the cup 54a to the external cervical os. The operator can then pull on the lavage device 10 to straighten the woman's uterus.

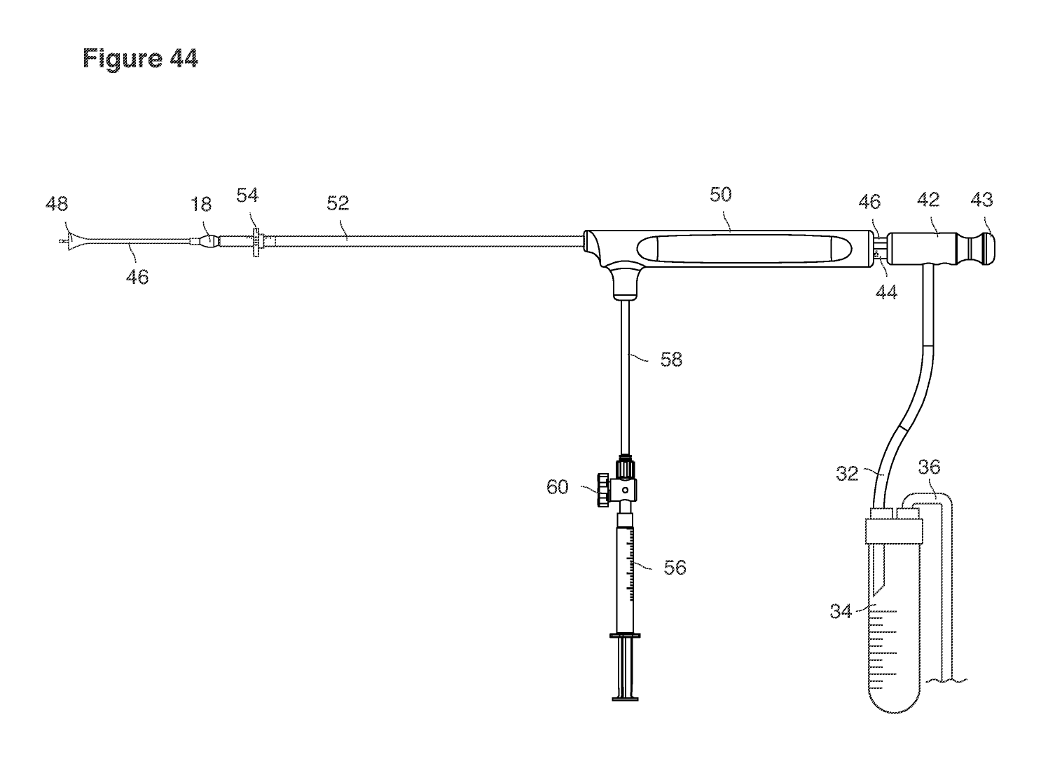

Referring to FIG. 44, rather than having the collection bottle 34 mounted to the cart 100, as shown in FIG. 16, the collection bottle 34 can hang off the device 10 with the suction line 36 running to the cart 100.

A number of embodiments of the invention have been described. Nevertheless, it will be understood that various modifications may be made without departing from the spirit and scope of the invention. Accordingly, other embodiments are within the scope of the following claims.

What is claimed is:

1. A device for recovering one or more blastocysts from a uterus of a human, comprising:
an outer guide member for insertion into a cervical canal of the human, the outer guide member including a distal portion with an activatable seal for isolating the uterus from the external environment, the outer guide member defining a lumen having a longitudinal axis; and
an inner catheter located within the lumen and slidable along the longitudinal axis of the lumen relative to the outer guide member, the inner catheter having a distal tip positionable distally of the seal to extend into the uterus, the inner catheter including a fluid delivery lumen terminating at a distal fluid delivery port for delivering fluid into the uterus and a flared suction port for aspirating fluid and entrained blastocysts from the uterus.

2. The device of claim 1, wherein the inner catheter includes a funnel that defines the flared suction port.

3. The device of claim 2, wherein the funnel is configured to expand outward when the inner catheter is extended distally relative to the outer guide member.

4. The device of claim 1, wherein a cross-sectional area of the flared suction port is larger than a cross-sectional area of the outer guide member.

5. The device of claim 1, wherein the flared suction port is located between the seal and the distal fluid delivery port.

6. The device of claim 1, wherein the flared suction port is defined around the fluid delivery lumen.

7. The device of claim 1, wherein the inner catheter is steerable.

8. The device of claim 7, wherein the inner catheter includes a memory-retaining material, and wherein, upon extension into the uterus, the inner catheter is configured to be steered by bending according to a pre-bent shape of the memory-retaining material.

9. A system for recovering one or more blastocysts from a uterus of a human, comprising:
a device, comprising:
an outer guide member for insertion into a cervical canal of the human, the outer guide member including a distal portion with an activatable seal for isolating the uterus from the external environment, the outer guide member defining a lumen having a longitudinal axis; and
an inner catheter located within the lumen and slidable along the longitudinal axis of the lumen relative to the outer guide member, the inner catheter having a distal tip positionable distally of the seal to extend into the uterus, the inner catheter including a fluid delivery lumen terminating at a distal fluid delivery port for delivering fluid into the uterus and a flared suction port for aspirating fluid and entrained blastocysts from the uterus; and
a controller programmed to cyclically deliver lavage liquid to the uterus via the fluid delivery lumen and apply vacuum to the device from a vacuum source remote from the device.

10. The system of claim 9, wherein the controller includes a pump for delivering the lavage liquid and a pump for applying the vacuum.

11. The system of claim 9, wherein the controller includes electro-mechanical means for controlling the delivery of lavage fluid and the application of vacuum.

12. The system of claim 9, wherein the controller is programmed to cyclically deliver varying amount of lavage liquid.

13. The system of claim 9, further including a lavage fluid bag for supplying the lavage liquid.

14. The system of claim 9, further including an embryo recovery trap for receiving the aspirated fluid and entrained blastocysts.

15. A process for recovering one or more blastocysts from a uterus of a human, comprising:
placing a device trans-vaginally into the cervical canal, the device including an outer guide member and an inner catheter located within the outer guide member, the outer guide member including a seal for isolating the uterus from the external environment;
advancing the inner catheter relative to the outer guide member positioning a distal region of the inner catheter within the uterus;
delivering fluid through the inner catheter to the uterus; and
applying a vacuum to the uterus to aspirate fluid and entrained blastocysts from the uterus through a flared suction port.

16. The process of claim 15, wherein placing the device includes locating the seal in the cervical canal.

17. The process of claim 15, wherein locating the seal includes locating the seal between the internal cervical os and the external cervical os such that the seal does not extend into the vagina or the uterus.

18. The process of claim 15, wherein advancing the inner catheter includes mechanically agitating an inner surface of the uterus.

19. The process of claim 15, wherein advancing the inner catheter includes positioning the distal region of the inner catheter proximate an inner wall of the uterus.
